(19) United States
(12) Patent Application Publication
(10) Pub. No.: US 2026/0097043 A1
(43) Pub. Date: Apr. 9, 2026

(54) NOVEL FORMULATIONS AND VEHICLES

(71) Applicant: WisTa Laboratories Ltd., Singapore (SG)

(72) Inventors: Yin Sze Loh, Singapore (SG); John Mervyn David Storey, Old Aberdeen, Aberdeenshire (GB); Claude Michel Wischik, Aberdeen, Aberdeenshire (GB); Björn Olaf Schelter, Aberdeen, Aberdeenshire (GB)

(73) Assignee: WisTa Laboratories Ltd., Singapore (SG)

(21) Appl. No.: 19/113,777

(22) PCT Filed: Sep. 20, 2023
(86) PCT No.: PCT/EP2023/075950

§ 371 Date: Mar. 20, 2025

(30) Foreign Application Priority Data
Sep. 21, 2022 (SG) 10202251102T

(51) Int. Cl. A61K 31/5415; A61J 1/03; A61J 1/18; A61K 9/20; A61K 9/28; A61K 9/48

(52) U.S. Cl. CPC A61K 31/5415; A61J 1/035; A61J 1/18; A61K 9/2013; A61K 9/2018; A61K 9/2027; A61K 9/2054; A61K 9/2095; A61K 9/2813; A61K 9/282; A61K 9/2853; A61K 9/2893; A61K 9/485; A61K 9/4858; A61K 9/4866; A61K 9/4883; A61K 9/4891; A61J 2205/30

(57) ABSTRACT

The invention provides different types of floating composition of diaminophenothiazine (DAPTZ) compounds such as methylene blue, which provide rapid-release in the stomach, or are gastro-retained. They further provide methods of making and using the same in the treatment and/or prevention of diseases. In one embodiment the invention provides an oral pharmaceutical composition which is a film-coated solid tablet comprising a DAPTZ compound as active ingredient in a gastro-retentive platform. In another embodiment the invention provides an oral pharmaceutical composition comprising an elongate hollow cylindrical capsule containing a weighting agent which is retained at one end, the capsule being buoyant and self-orientating, wherein the capsule is coated with an inner sustained release layer comprising a DAPTZ compound as active ingredient, and a further protective outer layer.

### CROSS-REFERENCE TO RELATED APPLICATIONS

This patent application claims the benefit of priority of application number SG10202251102T filed 21 Sep. 2022 and which is herein incorporated in its entirety.

### TECHNICAL FIELD

The present invention relates generally to compositions of 3,7-diaminophenothiazines (DAPTZ) having optimised pharmacokinetic properties, and their use in treating disease, for example neurodegenerative disease in humans.

### BACKGROUND TO THE INVENTION

Methylthioninium chloride (MTC) [Methylene Blue: 3,7-bisdimethylaminophenazothionium chloride, C₁₆H₁₈ClN₃S, 319.85 g/mol] was prepared for the first time in 1876 (The Merck Index, 13^{th} edition, Merck & Co., Inc., 2001, entry 6085).

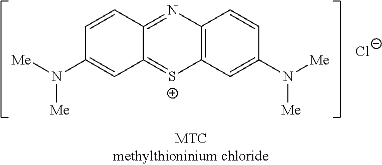

Various utilities have been described for MTC including use as a medical dye, as a redox indicator, an antiseptic, for the treatment and prevention of kidney stones, the treatment of melanoma, malaria, viral and microbial infections, and Alzheimer's and other neurodegenerative disease. MTC has also been used as an oxidizing agent and as an antidote in the case of CO, nitrite and aniline poisoning.

MT is a redox molecule and, depending on environmental conditions (e.g., pH, oxygen, reducing agents), exists in equilibrium between a reduced [leucomethylthioninium (LMT) or hydromethylthioninium (HMT)] and oxidized form methylthioninium (MT⁺).

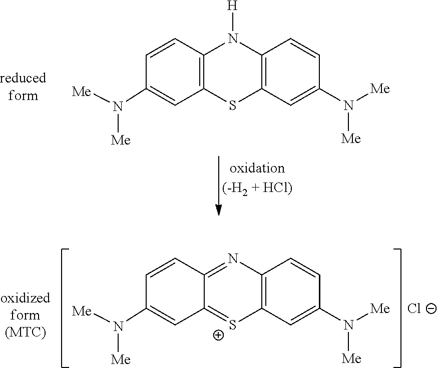

The “reduced form” (or “leuco form”) is known to be unstable and can be readily and rapidly oxidized to give the corresponding “oxidized” form.

WO96/30766 describes MT containing compounds for use in the treatment and prophylaxis of various diseases, including AD and Lewy Body Disease.

WO2012/072977 relates to solid dosage forms of MTC and to methods of preparing such solid dosage forms. This publication discloses, inter alia, tablet formulations in which the polymorphic form of the active ingredient is stable.

A preliminary pharmacokinetic model for methylene blue, based on studies of urinary excretion data sets in humans, dogs and rats, was proposed by DiSanto and Wagner, J Pharm Sci 1972, 61:1086-1090 and 1972, 61:1090-1094 and Moody et al., Biol Psych 1989, 26: 847-858.

Peter et al. (2000) Eur J Clin Pharmacol 56: 247-250 provided a model which integrated blood level data, which contradicted the earlier data from DiSanto and Wagner as regards terminal elimination half-life.

May et al. (Am J Physiol Cell Physiol, 2004, Vol. 286, pp. C1390-C1398) showed that human erythrocytes sequentially reduce and take up MTC i.e. that MTC itself is not taken up by the cells but rather that it is the reduced from of MT that crosses the cell membrane. They also showed that the rate of uptake is enzyme dependent; and that both oxidised and reduced MT are concentrated in cells (reduced MT re-equilibrates once inside the cell to form oxidised MT).

Based on these and other disclosures, it is believed that orally administered MTC and similar drugs are taken up in the gut and enter the bloodstream, with unabsorbed drug percolating down the alimentary canal, to the distal gut (colon). One important undesired side-effect is the effect of the unabsorbed drug in the distal gut, for example, sensitisation of the distal gut and/or antimicrobial effects of the unabsorbed drug on flora in the distal gut, both leading to diarrhoea.

Since it is the reduced form of MT that is taken up by cells, it has been proposed to administer a reduced- or leuco-form to patients. This may also reduce reliance on the rate limiting step of enzymatic reduction.

WO2009/044127 discloses that MTC in a clinical trial context had two systemic pharmacological actions: cognitive effects and haematological effects, but that these actions were separable. Specifically the cognitive effects did not show a monotonic dose-response relationship, whereas the haematological effects did. It was proposed that two distinct species were responsible for the two types of pharmacological activity: MTC absorbed as the uncharged Leuco-MT form being responsible for the beneficial cognitive activity, and MTC absorbed as an oxidised dimeric species being responsible for the oxidation of haemoglobin.

WO2009/044127 described how dosage forms could be used to maximise the bioavailability of the therapeutically active (cognitively effective) species whether dosing with oxidised or leuco-DAPTZ compounds (such as the leuco compounds of WO 02/055720, WO2007/110627 and WO2012/107706). For example WO2009/044127 described how, particularly for oxidized DAPTZ compounds, it may be desirable that the dissolve rapidly in the stomach (e.g. releases at least 50% of active ingredient within 30 minutes under standard conditions) so as to avoid percolation to the gut. It is further described how, particularly for oxidized DAPTZ compounds, it may be desirable that they be provided as gastroretained formulations for the same reason. It is further described how, since the same limitations in absorption do not apply to reduced/leuco-DAPTZ as apply to oxidised compounds, it may that these are the preferred form for use as slow or delayed release formulations.

WO 02/055720 discloses the use of reduced forms of certain diaminophenothiazines for the treatment of protein aggregating diseases, primarily tauopathies.

WO2007/110627 disclosed certain 3,7-diamino-10H-phenothiazinium salts to be effective as drugs or pro-drugs for the treatment of diseases including Alzheimer's disease. These compounds are also in the “reduced” or “leuco” form when considered in respect of MTC. These leucomethylthioninium compounds were referred to as “LMTX” salts.

WO2012/107706 described other LMTX salts having superior properties to the LMTX salts listed in WO2007/110627, including leuco-methylthioninium bis(hydromethanesulfonate) (LMTM):

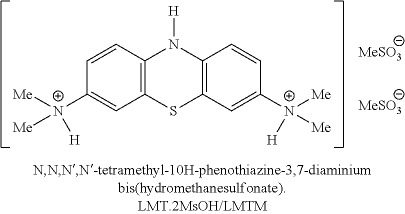

LMTM has superior pharmaceutic properties in terms of solubility and pKa, and is not subject to the absorption limitations of the MT⁺ form (Baddeley et al., 2015).

WO2018/019823 describes novel regimens for treatment of neurodegenerative disorders utilising methylthioninium (MT)-containing compounds. Briefly, these regimens identified two key factors. The first was in relation to the dosage of MT compounds, and the second was their interaction with symptomatic treatments based on modulation of acetylcholinesterase levels.

In the analysis described in WO2018/019823, low doses of MT compounds (for example 4 mg b.i.d.) showed therapeutic benefits when monotherapy was compared against add-on. The efficacy profiles were similar in mild and moderate subjects for most of the measured outcomes.

Based on analyses, and given that lower doses (4 mg twice a day) had a better overall clinical profile than the high dose (100 mg twice a day), WO2018/019823 teaches methods of treatment of neurodegenerative disorders of protein aggregation which comprise oral administration of MT-containing compounds, wherein said administration provides a total of between 0.5 and 20 mg of MT to the subject per day, optionally as a single dose or split into 2 or more doses. For a given daily dosage, WO2018/019823 teaches that more frequent dosing will lead to greater accumulation of a drug.

More recently, WO2020/020751 described a novel pharmacokinetic (PK) model for dosing LMT compounds in patient populations. As expected, there was substantial variability in the MT Cₘₐₓ values across the population for the given low dosage. Analysis of the distribution confirmed the findings in WO2018/019823 that low dosages (4 mg MT b.i.d.) were efficacious (as measured, for example, by reduced decline on the Alzheimer's Disease Assessment Scale-cognitive subscale (ADAS-cog). It further confirmed that monotherapy gave a substantial benefit by this criterion compared to add-on therapy with AChEIs and/or memantine (with the mean benefit of between monotherapy and add-on being ˜4 ADAS-cog units over 65 weeks).

However, unexpectedly in view of the previously described lack of any recognisable dose response, the analysis in WO2020/020751 revealed that there exists a concentration response within the low dose treated population. These insights suggested that it was advantageous to adopt a dosing regimen which both maximises the proportion of subjects in which the MT concentration will exceed the Cₘₐₓ or Cₐᵥₑ threshold, and also maximises the expected therapeutic efficacy of LMTM whether it is taken alone or in combination with (or at least preceded by) symptomatic treatments, while nevertheless maintaining a relatively low dose so as to maintain a desirable clinical profile in relation to being well tolerated with minimal side-effects. WO2020/020751 suggests that the minimum dose which achieves all these objectives is at least 20 mg/day, and doses in the range 20-40 mg/day, or 20-60 mg/day would be expected to maximise the therapeutic benefit, although good efficacy, particularly in AD patients not pre-treated with symptomatic treatments, can still be seen at dosages of 100 mg or more.

Prior-filed PCT/EP2023/064369 which is herein incorporated in its entirety describes how a 4 mg twice weekly dose of MTC showed therapeutic benefits in treating Alzheimer's diseases.

Although WO2009/044127 discussed above represented an important contribution to the art in relation to preferred dosage formats, and certain examples of such forms were provided it can be seen that providing novel formulations which optimise the dosing of DAPTZ compounds would provide a useful contribution to the art.

### DISCLOSURE OF THE INVENTION

The present inventors have provided different floating compositions of DAPTZ compounds which provide rapid-release in the stomach, or are gastro-retained. They further provide methods of making and using the same in the treatment and/or prevention of diseases.

In embodiments the novel compositions are adapted for release of the DAPTZ drug in the stomach, to avoid the DAPTZ drug percolating to the small intestine where its uptake and cognitive benefit may be impaired, and associated side effects may be reduced. Release in the acidic stomach maximises reduction due to the acidic ambient environment and presence of reductive enzymes and other reductases (e.g. glutathione). It also minimises potential MT⁺ side effects. In addition, the reduced form of the molecule is most stable at low pH thus facilitating absorption at this site

In embodiments the novel compositions are controlled or sustained release formulations which allow for less frequent dosing and hence increased compliance. Particularly In dementia management, patient compliance is a very relevant issue in the socioeconomic impact of a drug regime as we deal with patients with cognitive impairment (see Kanasty, Rosemary, et al. “A pharmaceutical answer to nonadherence: Once weekly oral memantine for Alzheimer's disease.” Journal of Controlled Release 303 (2019): 34-41.).

The Examples herein demonstrate the improved efficacy of gastroretained dosage forms of the invention in an animal model of a bacterial disease which can be treated with a DAPTZ compound (MTC). Further Examples indicate unexpected efficacy for rapid release MTC dosage forms, which the present inventors have demonstrated are capable of flotation.

In the light of these results, and the other disclosure herein, it can be seen that gastroretained dosage forms such as those which float while rapidly releasing the DAPTZ compound, or which are gastroretained providing sustained release, will have utility in the treatment of diseases in which DAPTZ compounds have shown efficacy, such as neurodegenerative disease.

Although a variety of methylene blue-based formulations are known in the art, generally speaking these have not been targeted for dissolution in the stomach, and indeed many are targeted specifically to the large or small intestine (see e.g. Repici, Alessandro, et al. “Efficacy of per-oral methylene blue formulation for screening colonoscopy.” Gastroenterology 156.8 (2019): 2198-2207; Tariq, Bina, et al. “Evaluating the safety of oral methylene blue during swallowing assessment: a systematic review.” European Archives of Oto-Rhino-Laryngology (2021): 1-15.)

Floating drug delivery systems are also known in the art (see e.g. Patel, Nirav, et al. “Floating drug delivery system: an innovative acceptable approach in gastro retentive drug delivery.” Asian Journal of Pharmaceutical Research 2.1 (2012): 7-18). However, it remains the case that providing suitable systems must be done case by case basis, dependent on the active pharmaceutical ingredient (API).

As explained above, MTC and LMTM give rise to two different forms of active MT moieties, the positively charged oxidized form and the neutrally charged reduced (leuco) form of the MT moieties respectively. The MT moieties are the active moieties that are absorbed by the stomach and intestines to enter the blood stream. It is to be appreciated that the oxidized positively charged MT form is blue in colour as compared to the reduced colourless neutrally charged leuco form.

In various embodiments, the DAPTZ compound is selected from methylthioninium chloride (MTC) and leuco-methylthioninium (bis)mesylate (LMTM). Other MT and LMT compounds are described hereinafter.

In various embodiments, the formulation comprises methylthioninium chloride (MTC) and gastro-retentive platform.

In various embodiments, the formulation comprises leuco-methylthioninium (bis)mesylate (LMTM) and gastro-retentive platform.

In various embodiments, the formulation comprises methylthioninium chloride (MTC), leuco-methylthioninium (bis)mesylate (LMTM) and a gastro-retentive platform.

Therefore in various embodiments of the invention there are provided oral formulations comprising a diaminophenothiazine compound and a gastro-retentive platform as described herein. As explained hereinafter these may be used a sustained/controlled release targeting upper gastrointestinal tract, for example for treatment and/or prevention of disease, for example a neurodegenerative disorder of protein aggregation, or a disease of gastric perforations or ulcerations of an upper gastrointestinal tract caused by an infective agent.

In certain embodiments, the formulations may be delivered by nasogastric tube, as explained below.

These and other embodiments will now be described in more detail:

Thus in one aspect there is provided a pharmaceutical composition (e.g. an oral dosage unit) which is a solid tablet comprising a diaminophenothiazine (DAPTZ) compound as active ingredient in a gastro-retentive platform,
wherein the DAPTZ compound is selected from an oxidized methylthioninium (MT⁺) compound or a leucomethylthioninium (LMT) compound or a combination thereof,
wherein the formulation comprises a rapid release matrix including a hydrophilic macromolecule and is adapted to float in the stomach,
where in the formulation further comprises one or more other accompanying active ingredients, additives, excipients, diluents, binders, lubricants, disintegrators, fillers, stabilizers, surfactants, antioxidants, or combinations thereof.

Such compositions are capable of floating in the stomach (i.e. being buoyant in human gastric fluid). This means that they are retained in the stomach longer than comparable formulations which immediately sink (and therefore more quickly exit through the pyloric opening into the small intestine, or release the DAPTZ compound there). This maximises release of the active DAPTZ compound within the gastric cavity, where the DAPTZ compound may be preferably absorbed in an acidic environment.

For example a tablet of the invention may have a composition having a floating time of at least 120 seconds when placed on deionised water, more preferably 2, 3, 4 or 5 minutes.

Such compositions may preferably not have bioadhesive properties, and may preferably not be a swelling system.

Example compositions according to this aspect of the invention were capable of disintegration in a similar timescale to the floating time (c. 2 to 3 minutes in each case). It is believed that this is advantageous in ensuring appropriate release of the API.

Preferably the tablet has a disintegration time of less than 5 minutes, preferably less than 4 minutes, or less than 3 minutes.

Preferably the tablet has a disintegration time of a similar order of magnitude to the floating time, preferably between 200% to 50% of the floating time.

Preferably the amount of methylthioninium or leucomethylthioninium in the platform is equal to or between 1 and 10 mg, more preferably about 2 to 6 mg, more preferably about 2 to 5 mg, more preferably about or equal to 4 mg.

In some embodiments, the amount is about 1, 1.5, 2, 2.5, 3, 3.5, 4, 5, 6, 7, 8, 9, 10, 15, 20, 25, 30 mg of MT.

Unless context demands otherwise, where the term “MT” is used herein e.g. in relation dosages or loading or release, it is used as shorthand for methylthioninium (MT/MT⁺) or leuco-methylthioninium (LMT), the difference in molecular weight of these molecules being negligible in relation to recited dosages or loadings. Thus “MT” in that context will be understood to apply mutatis mutandis to “MT⁺” or “LMT”

In some embodiments, the amount of MT in the composition is 0.1 to 10 mg.

In some embodiments, the amount of MT in the composition is 0.05 to 10 mg.

In some embodiments, the amount of MT in the composition is 0.05 to 5 mg.

An example composition may contain 1 to 10 mg of MT.

A further example composition may contain 2 to 9 mg of MT.

A further example composition may contain 3 to 8 mg of MT.

A further preferred composition may contain 3.5 to 7 mg of MT.

A further preferred composition may contain 4 to 6 mg of MT.

In some embodiments, the amount is about 0.05, 0.1, 0.5, 1, 1.5, 2, 2.5, 3, 3.5, 4, 5, 6, 7, 8, 9, 10 mg of MT.

Using the weight factors described or explained herein, one skilled in the art can select appropriate amounts of an MT-containing compound to use in the formulations.

For example, as explained below, the MT weight factor for LMTM is 1.67. Since it is convenient to use unitary or simple fractional amounts of active ingredients, non-limiting example LMTM dosage units may include 2, 2.5, 3, 3.5, 4, 5, 6, 7, 8, 9, 10, 15, 16, 17, 18 mg etc.

As explained below, the MT weight factor for MTC·5H₂0 is 1.44. Since it is convenient to use unitary or simple fractional amounts of active ingredients, non-limiting example MTC·5H₂0 dosage units may include 1.5, 2, 2.5, 3, 3.5, 4, 5, 6, 7, 8, 9, 10, 15, 18, 20 mg etc.

Typically the DAPTZ compound is formulated with an excess e.g. at least or equal to 2 to 3 times an amount of the gastro-retentive platform.

Preferably the tablet is generally circular with a diameter of about 4 to 6 mm, preferably about 5 mm, and a thickness of 2 to 3 mm e.g. 2.2 to 2.6 mm.

As those skilled in the art will appreciate, tablets of this size may be used not only orally, but also delivered by nasogastric tube, for example to individuals who are unable to swallow safely (Zhu L L, Xu L C, Wang H Q, Jin J F, Wang H F, Zhou Q. Appropriateness of administration of nasogastric medication and preliminary intervention. Ther Clin Risk Manag. 2012; 8:393-401. doi: 10.2147/TCRM.S37785. Epub 2012 Nov. 20. PMID: 23185120; PMCID: PMC3506154.)

Preferably the tablet has a hardness of 3 to 9 kp, preferably about 6 to 7 kp.

Most preferably the tablet has a total weight of equal to or between 30 and 60 mg, more preferably 40 and 60 mg, 50 and 60 mg, e.g. about 53 mg, 54 mg, 55 mg, 56 mg, 56 mg, 58 mg, 59 mg, 60 mg.

Preferably the tablet has a density of <1.5 g/cm³ e.g. about 0.8 to 1.5 e.g. about 1.0, 1.1, 1.2, 1.3 or 1.4. e.g. about 1.2, 1.3 or 1.4.

Most preferably the platform includes a coating which is a hydrophilic macromolecule.

Optionally the hydrophilic macromolecule is Poly(vinyl alcohol) (PVA). Optionally the PVA is part-hydrolysed. Optionally the coating further comprises one or more (e.g. all of) talc, titanium dioxide, macrogol PEG 3350, lecithin, colouring.

Optionally the colouring is FD&C blue #2/indigo carmine aluminium lake (dialuminum;2-(3-hydroxy-5-sulfonato-1H-indol-2-yl)-3-oxoindole-5-sulfonate).

Optionally the coating is an Opadry® II coating (see e.g. Koo, Otilia M Y, et al. “Investigation into stability of poly (vinyl alcohol)-based Opadry® II films.” AAPS Pharm Sci Tech 12 (2011): 746-754.). Optionally the coating is Opadry® II Blue 85G205011. These are available from Colorcon as coating formulations containing excipients which serve as coating aids. Opadry® formulations may also contain talc, polydextrose, triacetin, polyethyleneglycol, polysorbate 80, titanium dioxide, and one or more dyes or lakes.

Most preferably the coating is about 5 to 8% of the total tablet weight.

Coating of the tablets may conveniently be carried out using a coating pan or more preferably a rotary spray coater. Preferably the coating is applied by spray-coating the tablet core with aqueous coating until the desired weight gain has been achieved, with the tablet cores being optionally pre-warmed to between 42-52° C., and adjusting temperature of inlet air so the exhaust temperature is maintained between 42-52° C.

Suitable carriers, diluents, excipients, etc. can be found in standard pharmaceutical texts. See, for example, Handbook of Pharmaceutical Additives, 2nd Edition (eds. M. Ash and I. Ash), 2001 (Synapse Information Resources, Inc., Endicott, New York, USA), Remington's Pharmaceutical Sciences, 20th edition, pub. Lippincott, Williams & Wilkins, 2000; and Handbook of Pharmaceutical Excipients, 2nd edition, 1994.

Most preferably the platform includes a diluent such as mannitol and/or microcrystalline cellulose.

Most preferably the platform includes a disintegrant such as crospovidone.

Most preferably the platform includes a lubricant such as magnesium stearate.

Preferably the tablet has a friability of less 1%.

Thus in various embodiments, the gastro-retentive platform can include a gastro-targeting rapid release platform to ensure the release of the active DAPTZ compound/s happens within the gastric cavity where DAPTZs are preferably absorbed in an acidic environment.

In various embodiments, the rapid release matrix may be formed of elements that dissolve in low pH allowing any DAPTZ loaded microparticle embedded within the matrix to be disbursed throughout and held in place within the matrix but not released until it reaches the stomach. The lower pH in the stomach then allows for a rapid release of the DAPTZ loaded microparticles within the stomach.

These properties can help to optimize controlled gastric absorption of DAPTZ compounds overtime and reduce side effects of the drug on the lower large intestines. These properties can help to reduce dosing frequency of the said formulation.

“Hardness” (e.g. in kp, or “kilopond”) may be measured by standard methods known in the art—see e.g. Example 5.

“Density” may be measured by standard methods known in the art—see e.g. Example 5.

“Friability” may be measured by standard methods known in the art—see e.g. Example 5.

A “floating” composition or dosage unit as the term is used herein means the dosage unit is capable of floating when placed on deionised water for at least 60, preferably at least 120, or 150 seconds, or 1, 2, 3, 4, or 5 minutes (the “floating time”). The floating time of the buoyant capsules of the invention is considerably longer e.g. at least 1 hour, 2, 3, 4, 5, 6, 7, 8, 9, 10 hours.

“Disintegration time” may be measured by standard methods known in the art. Examples methods are provided herein.

“Dissolution time” may be measured by standard methods known in the art. Examples methods are provided herein. An “immediate release” product allows the ingredient or active moiety to dissolve in the gastrointestinal tract, without causing any delay or prolongation of the dissolution or absorption of the drug. Requirements for dissolution testing of immediate release products are set out in the Guidance for Industry (CDER 1997) “Dissolution testing for immediate release solid oral dosage forms”, (CDER 1997) “Immediate release solid oral dosage forms—Scale up and Post approval Changes”, ICH Guidance Q6A, Specifications: Test Procedures and Acceptance Criteria For New Drug Substances And New Drug Products. The most commonly employed dissolution test methods as described in the USP and European Pharmacopeia (6th edition) are the basket method (USP 1) and the paddle method (USP 2). The described methods are simple, robust, well standardized, and used worldwide. They are flexible enough to allow dissolution testing for a variety of drug products. The following parameters influencing the dissolution behaviour may for example be relevant for selecting the appropriate in vitro dissolution test conditions for an immediate release solid oral product: apparatus, stirring speed, dissolution medium and temperature. Because of the biopharmaceutical properties of MTC and its expected desirable absorption characteristics in the upper gastrointestinal tract, it was preferable to produce rapidly dissolving tablets of MTC.

Compositions according to the invention can be dissolution tested in a USP-2 apparatus in 900 ml of 0.1 N HCl, with paddles rotating at 50-75 rpm. Compositions according to the invention exhibit at least the acceptance criteria cited for Stage 1 (S1) testing in the USP 32 (The United States Pharmacopeia, edited by the United States Pharmacopeial Convention, Inc., 12601 Twinbrook Parkway, Rockville, MD 20852; Published by Rand McNally, Inc., 32nd Edition, 2008).

Thus in various embodiments, the formulation comprises a DAPTZ compound as described herein and a rapid release matrix as the gastro-retentive platform, for example as described above.

In various embodiments, the formulation comprises methylthioninium chloride (MTC) and a rapid release matrix as the gastro-retentive platform, for example as described above.

In various embodiments, the formulation comprises leuco-methylthioninium (bis)mesylate (LMTM) and a rapid release matrix as the gastro-retentive platform.

In various embodiments, the formulation comprises methylthioninium chloride (MTC) leuco-methylthioninium (bis)mesylate (LMTM) and a rapid release matrix as the gastro-retentive platform.

Without wishing to be bound by theory, it is understood that, based on the determinations made by the present inventors, tablets having the qualities defined above can be easily swallowed without oro-dissolution (or buccal staining) but then have the capacity to float at the surface of the gastric fluid, with the low pH permitting rapid release of the DAPTZ compound, which leads to the released DAPTZ compound being maximally exposed to maximal surface area of the stomach mucosal lining in the acidic stomach environment, thereby providing optimal gastro-targeted absorption.

Examples of preferred DAPTZ compounds comprising MT/LMT are described hereinafter.

In one embodiment, the DAPTZ compound is MTC. Preferably the MTC tablet has the MTC composition shown in Table 1

In another embodiment the DAPTZ compound is LTMM. Preferably the LMTM tablet has the LMTM composition shown in Table 2.

Processes for providing the rapid-release floating tablets of the invention are described hereinafter.

Thus on one aspect, the invention provides a process for the manufacture of the tablet described herein, which process comprises:
(i) compression or granulation of the DAPTZ compound with one or more other accompanying active ingredients, additives, excipients, diluents, binders, lubricants, disintegrants, fillers, stabilizers, surfactants, antioxidants, if present;
(ii) applying the film coating to the tablets.

Optionally the step of applying a film coating is carried out by spray-coating the tablet core in a rotary tablet coater, wherein the coating coater and cores are optionally pre-heated to 42-52° C., and adjusting temperature of inlet air so the exhaust temperature is maintained between 42-52° C.

In other aspects of the invention, the gastro-retained formulation is a sustained/controlled release composition, resulting in the release of the active ingredient of DAPTZ targeting the upper gastrointestinal tract over a period of time.

In particular, in light of the Examples 1 to 4 showing evidence of the effectiveness of gastro-targeted sustained release MTC formulations in treating disease in animals, and Examples 5 to 7 showing the effectiveness of MTC mini-tablets which the inventors have divined are capable of floatation, further floating gastro-retained platforms for administering DAPTZ compounds were developed. These were based generally on the dosage forms described in WO2012/004231 (Jagotec AG) which is specifically incorporated herein by cross reference, and available from Skyepharma Production S.A.S, Z. A. de Chesnes Ouest—55 Rue du Montmurier 38291 Saint-Quentin-Fallavier cedex, B.P. 45, France, but adapted specifically in the light of the insights of the present inventors into the specific pharmacokinetics and pharmacodynamic of MT and LMT compounds.

Thus in a further aspect of the invention there is provided an oral pharmaceutical composition comprising a diaminophenothiazine (DAPTZ) compound as active ingredient on a gastro-retentive platform,
wherein the DAPTZ compound is selected from an oxidized methylthioninium (MT) compound or a leucomethylthioninium (LMT) compound or a combination thereof,
   wherein the gastro-retentive platform is adapted to float in the stomach
   wherein the platform is a hollow capsule in the form of generally cylindrical shape having two opposing ends, wherein the capsule is weight biased such that it is heavier at one end than the other end,
   wherein the length of the capsule along its long axis is such that it is larger than the average diameter of the pyloric valve in humans.

Preferably, the dosage form is at least 12 mm in length and is more preferably 15 mm or greater along this axis e.g. between 20 and 24 mm. The upper limit for the length along this axis is determined by what is comfortable to be swallowed by a human patient.

Preferably, the dosage form in this dimension is not longer than about 30 or 31 mm.

Preferably, the dosage form has a diameter of less than 10 mm e.g. about 7, 8, or 9 mm.

In some embodiments, said capsules are gelatine capsules.

In some embodiments, said capsules are HPMC (hydroxypropylmethylcellulose) capsules.

In a related aspect there is provided an oral pharmaceutical composition comprising an elongate hollow cylindrical capsule which is impermeable to gastric fluid and having a fill volume inside containing a weighting agent which is retained at one end of the fill volume, the capsule being buoyant and self-orientating in an aqueous fluid such that it floats in the aqueous fluid with its long axis perpendicular to the surface of the aqueous fluid,
wherein the capsule is coated with a first inner sustained release layer comprising a diaminophenothiazine (DAPTZ) compound as active ingredient,
and wherein the first layer is coated with a second outer layer not including an active ingredient,
wherein the DAPTZ compound is selected from an oxidized methylthioninium (MT) compound or a leucomethylthioninium (LMT) compound or a combination thereof,
wherein the first and second layers each comprise at least one hydrophilic polymer and at least one hydrophobic polymer, plus optionally one or more lubricants, glidants or plasticisers.

Thus the first layer comprises the DAPTZ compound. This is “over-coated” with a top coating with a protective effect.

In one embodiment the compositions of the first and second layers are partly or wholly insoluble in aqueous solution, so they can be applied as an aqueous slurry to the capsule.

In one embodiment the first layer makes up 5 to 20% of the total weight of the composition.

In one embodiment the second layer makes up about 1 to 5% of the total weight of the final dosage form.

In one embodiment the hydrophilic polymer and the hydrophobic polymer are both water-insoluble acrylic copolymers, which are optionally acrylate/ammonium methacrylate copolymers.

In one embodiment the hydrophobic polymer is Eudragit® RS 30D. Eudragit® RS 30D is composed of poly(ethyl acrylate-co-methyl methacrylate-co-trimethylammonioethyl methacrylate chloride) 1:2:0.1.

In one embodiment the hydrophilic polymer is Eudragit® RL 30D. Eudragit® RS 30D is composed of poly(ethyl acrylate-co-methyl methacrylate-co-trimethylammonioethyl methacrylate chloride) 1:2:0.2.

In one embodiment the weight ratio of first layer hydrophobic polymer to hydrophilic polymer is about 4:1.

In one embodiment the weight ratio of second layer hydrophobic polymer to hydrophilic polymer is about 1:4.

The formulation of the invention may further comprise other accompanying active ingredients, additives, excipients, diluents, binders, lubricants, disintegrators, fillers, stabilizers, surfactants, antioxidants, or combinations thereof].

In one embodiment each layer further comprises a plasticizer, which is optionally a citrate ester, which is optionally TriEthyl Citrate (TEC).

In one embodiment each layer further comprises a lubricant or gluidant, which is optionally talc.

In one embodiment the inner capsule has a diameter of about 8.5 mm and a locked length of about 23.3 mm.

Optionally the capsule has a “pre-coating” which may be an enteric-coating. An enteric coating, being resistant to gastric fluid, will retain the integrity to the dosage form during the period of release of drug substance. Enteric coatings are known in the art. An enteric coating comprises a film-forming polymer, which is soluble in an aqueous medium of a pH of higher than 5 but not soluble in an aqueous medium of a pH of about 5 or less.

In one embodiment the weighting agent comprises barium sulfate. Other weighting agents may be selected for their high density as well as their physiological inertness e.g. dibasic calcium phosphate, iron oxide, iron, titanium dioxide, high density calcium carbonate etc. The weight of the weighting agent may range from 50 up to 600 mg. In the Examples herein the core weighted capsule had a weight of about 750 mg, prior to coating. In the Examples herein a size 3 capsule was fitted into a size 00 capsule, in order to position the weighting agent stably at one end.

In some embodiments, the amount of MT in the composition is 10 to 120 mg.

An example composition may contain between 10 to 20, 30, 40, 50, 60, 70, 80, 90, 100, 110, or 120 mg of MT.

A further example dosage composition may contain 50 to 100 mg of MT.

In some embodiments, the composition releases less than 10% of the DAPTZ compound in pH 1.2 USP buffer within 1 hour of adding to the buffer, and more than 50% within 8 hours. Testing conditions can be as described in more detail in Example 10 (37° C.±0.5° C.; speed of rotation 75±4 rpm).

In the light of the present disclosure it will be appreciated that the capsule-based compositions of the Examples may be adapted to release the MT payload over longer periods e.g. over 1, 2, 3 or even more days. Loading of MT will be adjusted accordingly.

Therefore in some embodiments, the composition releases less than 10% of the DAPTZ compound in pH 1.2 USP buffer within 1, 2, 3, 4 or 8 hours of adding to the buffer, and more than 80% within 24, 48 or 72 hours.

Processes for providing the coated capsulates of the invention are described hereinafter.

Suitable coating processes for producing compositions of the invention include coating pans and fluidized-bed coating equipment as described in Remington: The Science and Practice of Pharmacy, Lippincott Williams & Wilkins, 21st ed. (2005) (“Remington's”). In some embodiments, the coating solution is applied to a capsule using a spray coating technique. Either an air-less spray or an air spray coating technique can be used for film coating as described in Remington's. The use of a spray coating technique permits finely nebulized droplets of the coating solution to be delivered to the capsule surface. These techniques are ideal for commercial production because they ensure uniform coverage of capsules without them sticking together.

In some embodiments, a side-vented coating pan is used to apply the coating solution to the capsule by a spray coating technique. Suitable side-vented coating pans include the Accela-Cota (Thomas Engineering, Hoffman Estates, Ill.), the Fast Coater (O'Hara Manufacturing Ltd., Toronto Canada), the Hi-Coater (Vector Corp., Marion, Iowa), the Driacoater (Driam Metallprodukt, GmbH, Eriskirch, Germany), and the Pro Coater (Glatt Air Techniques, Ramsey, N.J.).

Thus on one aspect, the invention provides a process for the manufacture of the coated capsule described herein, which process comprises:
(i) providing the capsule, and pre-heating it to obtain a product temperature of 35-40° C.;
(ii) spraying the capsule with a filtered first layer coating suspension until the required weight gain is achieved;
(iii) drying the coated capsules (e.g. for 30 minutes) to form a sustained polymeric first layer film coating,
(iv) optionally waiting for up to 24 hours;
(v) pre-heating the coated capsules to obtain a product temperature of about 40° C.;
(vi) spraying the coated capsule with a second layer coating suspension until the required weight gain is achieved;
(vii) drying the coated capsules (e.g. for 20 minutes) to form a sustained polymeric second layer film coating,
(viii) cooling of the capsules until a product temperature of 30° C. was obtained.

Preferably each coating suspension is an aqueous suspension, optionally having the composition shown in Table 3. As explained in the Examples hereinafter, it is preferred to filter the aqueous suspension comprising the API e.g. through a 420 μm sieve.

Also provided is a pharmaceutical composition obtainable by any of the processes of manufacture described herein.

Thus it will be appreciated that in addition to the floating rapid-release compositions described in the first aspect, the term ‘gastro-retentive platform’ and its plural form can include frameworks that are able to retain an effective amount of DAPTZ compound within the stomach for longer e.g. 3 hours or more. In various embodiments the gastro-retentive platform may include frameworks that are able to retain an effective amount of DAPTZ compound within the stomach for 4 hours or more, 5 hours or more, 6 hours or more. In various embodiments the gastro-retentive platform may increase bioavailability of an effective amount of DAPTZ compound by more than 1.2 times, 1.3 times, 1.4 times, 1.5 times, 1.6 times, 1.7 times, 1.8 times, 1.9 time or more than 2 times.

The MT-containing compounds used in the present invention can contain MT in either reduced or oxidised form. The “MT” is the active ingredient, which is to say that it is present to provide the recited therapeutic effect. Specifically, the compounds may comprise either of the MT moieties described above. The MT moieties per se described above are not stable. They will therefore be administered as MT compounds—for example LMT or MT⁺ salts.

| Structure | 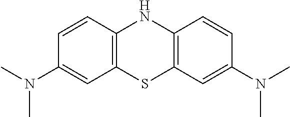 | 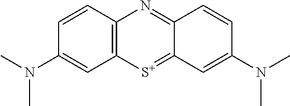 |
|---|---|---|
| IUPAC | N3,N3,N7,N7-tetramethyl-10H- | N3,N3,N7,N7- |
| | phenothiazine-3,7-diamine | tetramethylphenothiazin-5-ium- |
| | | 3,7-diamine |
| Composition | Formula Weight: 285.41(1) | Formula Weight: 284.40(1) |
| | Exact Mass: 285.1299683(1) | Exact Mass: 284.1215947(1) |
| | Formula: C₁₆H₁₉N₃S | Formula: C₁₆H₁₈N₃S |
| | Composition: C 67.33% H 6.71% | Composition: C 67.57% H |
| | N 14.72% S 11.23% | 6.38% N 14.78% S 11.27% |
| Synonyms | leucomethylthioninium (LMT) | oxidized methylthioninium (MT⁺) |
| | hydromethylthionine (HMT) | |

MT⁺ salts will generally include one or more anionic counter ions (X⁻) to achieve electrical neutrality. The compounds may be hydrates, solvates, or mixed salts of the MT⁺ salt.

LMT containing compounds will generally be stabilised, for example by the presence of one or more protic acids e.g. two protic acids.

The MT content of such salts can be readily calculated by those skilled in the art based on the molecular weight of the compound, and the molecular weight of the MT moiety. Examples of such calculations are given herein.

### LMT Compounds

In some embodiments, the MT compound is preferably an LMT compound.

In some embodiments, the MT compound is an “LMTX” compound of the type described in WO2007/110627 or WO2012/107706.

Thus, the compound may be selected from compounds of the following formula, or hydrates or solvates thereof:

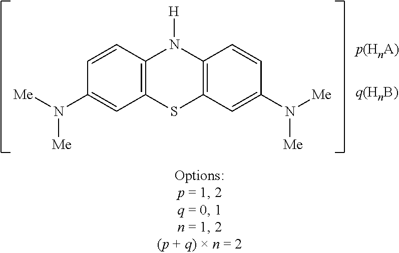

Each of HₙA and HₙB (where present) are protic acids which may be the same or different.

By “protic acid” is meant a proton (H⁺) donor in aqueous solution. Within the protic acid A⁻ or B⁻ is therefore a conjugate base. Protic acids therefore have a pH of less than 7 in water (that is the concentration of hydronium ions is greater than 10⁻⁷ moles per litre).

In one embodiment the salt is a mixed salt that has the following formula, where HA and HB are different mono-protic acids:

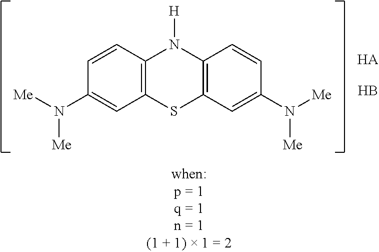

However preferably the salt is not a mixed salt, and has the following formula:

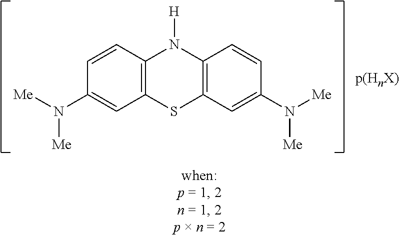

wherein each of HₙX is a protic acid, such as a di-protic acid or mono-protic acid.

In one embodiment the salt has the following formula, where H₂A is a di-protic acid:

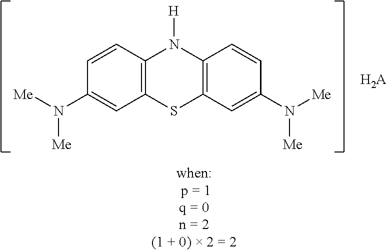

Preferably the salt has the following formula which is a bis monoprotic acid:

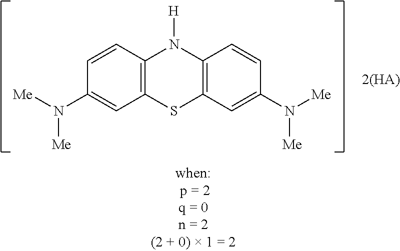

Examples of protic acids which may be present in the LMTX compounds used herein include:

Inorganic acids: hydrohalide acids (e.g., HCl, HBr), nitric acid (HNO₃), sulphuric acid (H₂SO₄)

Organic acids: carbonic acid (H₂CO₃), acetic acid (CH₃OOOH), methanesulfonic acid, 1,2-Ethanedisulfonic acid, ethanesulfonic acid, naphthalenedisulfonic acid, p-toluenesulfonic acid,

Preferred acids are monoprotic acid, and the salt is a bis(monoprotic acid) salt.

A preferred MT compound is LMTM:

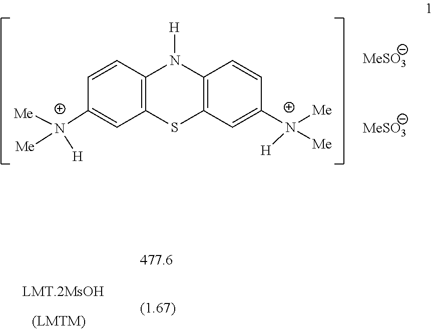

The anhydrous salt has a molecular weight of around 477.6. Based on a molecular weight of 285.1 for the LMT core, the weight factor for using this MT compound in the invention is 1.67. By “weight factor” is meant the relative weight of the pure MT containing compound vs. the weight of MT which it contains.

Other weight factors can be calculated for example MT compounds herein, and the corresponding dosage ranges can be calculated therefrom.

| | | | MW |
|---|---|---|---|
| | | | (weight |
| | Compound | Abbreviation | factor) |
| 2 | 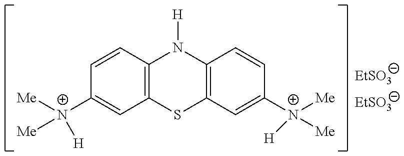 | LMT.2EsOH | 505.7 (1.77) |
| 3 | 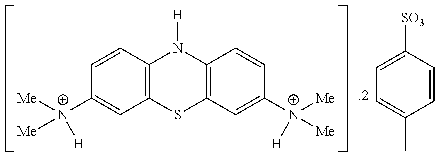 | LMT.2TsOH | 629.9 (2.20) |
| 4 | 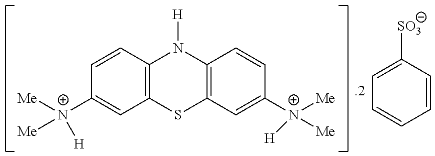 | LMT.2BSA | 601.8 (2.11) |
| 5 | 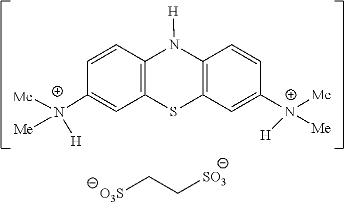 | LMT.EDSA | 475.6 (1.66) |
| 6 | 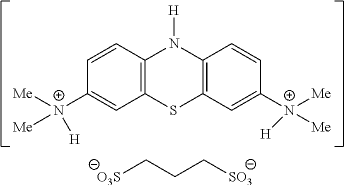 | LMT.PDSA | 489.6 (1.72) |
| 7 | 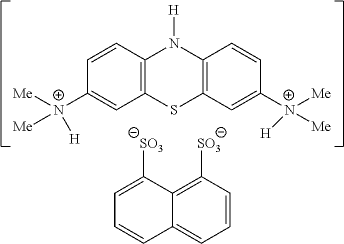 | LMT.NDSA | 573.7 (2.01) |
| 8 | 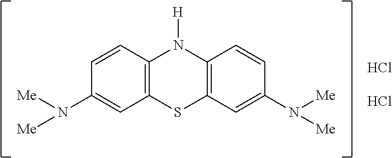 | LMT.2HCl | 358.33 (1.25) |

The dosages described herein with respect to MT thus apply mutatis mutandis for these MT containing compounds, as adjusted for their molecular weight.

### Oxidised MT Compounds

In another embodiment the MT compound is an MT⁺ compound.

Preferably the MT compound is an MT⁺ compound of the type described in WO96/30766 or WO2007/110630.

Thus, the compound may be selected from compounds of the following formula, or hydrates, solvates, or mixed salts thereof:

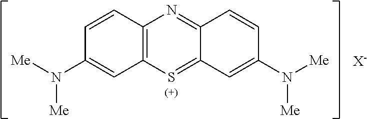

Where X⁻ is an anionic counter ion.

In some embodiments of the present invention the MT⁺ compound is MTC, for example a “high purity” MTC as described below.

In some embodiments of the present invention the MT⁺ compound is not MTC.

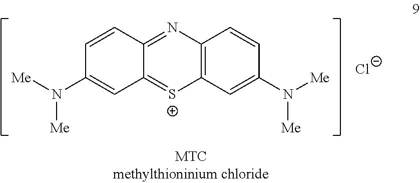

As explained in WO2011/036561 and WO2011/036558, MTC occurs in a number of polymorphic forms having different levels of hydration.

In some embodiments of the present invention, the MT⁺ compound is a high purity MTC.

In this context ‘high purity’ is defined by one or more of the criteria set out below.

In some embodiments, the MTC has a purity of greater than 97%.

In some embodiments, the MTC has a purity of greater than 98%.

In some embodiments, the MTC has a purity of greater than 99%.

In some embodiments, the MTC has less than 2% Azure B as impurity.

In some embodiments, the MTC has less than 1% Azure B as impurity.

In some embodiments, the MTC has less than 0.5% Azure B as impurity.

In some embodiments, the MTC has less than 0.1% Azure B as impurity.

In some embodiments, the MTC has less than 0.15% Azure A as impurity.

In some embodiments, the MTC has less than 0.10% Azure A as impurity.

In some embodiments, the MTC has less than 0.05% Azure A as impurity.

In some embodiments, the MTC has less than 0.15% Azure C as impurity.

In some embodiments, the MTC has less than 0.10% Azure C as impurity.

In some embodiments, the MTC has less than 0.05% Azure C as impurity.

In some embodiments, the MTC has less than 0.13% MVB (Methylene Violet Bernstein) as impurity.

In some embodiments, the MTC has less than 0.05% MVB as impurity.

In some embodiments, the MTC has less than 0.02% MVB as impurity.

All percentage purities recited herein are by weight unless otherwise specified.

In some embodiments, the MTC has an elementals purity that is better than that specified by the European Pharmacopeia (EP).

As used herein, the term ‘elementals purity’ pertains to the amounts of the twelve (12) metals specified by the European Pharmacopeia: Al, Cd, Cr, Cu, Sn, Fe, Mn, Hg, Mo, Ni, Pb, and Zn. The current edition of the European Pharmacopeia (8^{th} Edition, supplementum 8.8) specifies the following limits for these metals:

European Pharmacopeia 8.8 (EP8.8)

| Element | Maximum content (μg/g) |
|---|---|
| Aluminium (Al) | 100 |
| Cadmium (Cd) | 1 |
| Chromium (Cr) | 100 |
| Copper (Cu) | 300 |
| Tin (Sn) | 10 |
| Iron (Fe) | 200 |
| Manganese (Mn) | 10 |
| Mercury (Hg) | 1 |
| Molybdenum (Mo) | 10 |
| Nickel (Ni) | 10 |
| Lead (Pb) | 10 |
| Zinc (Zn) | 100 |

In one embodiment, the MTC has an elementals purity (e.g. for each of Al, Cd, Cr, Cu, Sn, Fe, Mn, Hg, Mo, Ni, Pb, and Zn) which is equal to or better than (i.e. lower than) the EP8.8 values set out in the table above.

In one embodiment, the MTC has an elementals purity which is equal to or better than 0.9 times the EP8.8 values set out in the table above.

In one embodiment, the MTC has an elementals purity which is equal to or better than 0.8 times the EP8.8 values set out in the table above.

In one embodiment, the MTC has an elementals purity which is equal to or better than 0.7 times the EP8.8 values set out in the table above.

In one embodiment, the MTC has an elementals purity which is equal to or better than 0.5 times the EP8.8 values set out in the table above.

(For example, 0.5 times the EP8.8 values as set out above are 50 μg/g Al, 0.5 μg/g Cd, 50 μg/g Cr, etc.)

In one embodiment the MTC has a chromium level that is equal to or better than (i.e. lower than) 100 μg/g.

In one embodiment the MTC has a chromium level that is equal to or better than (i.e. lower than) 10 μg/g.

In one embodiment the MTC has a copper level that is equal to or better than (i.e. lower than) 300 μg/g.

In one embodiment the MTC has a copper level that is equal to or better than (i.e. lower than) 100 μg/g.

In one embodiment the MTC has a copper level that is equal to or better than (i.e. lower than) 10 μg/g.

In one embodiment the MTC has an iron level that is equal to or better than (i.e. lower than) 200 μg/g.

In one embodiment the MTC has an iron level that is equal to or better than (i.e. lower than) 100 μg/g.

All plausible and compatible combinations of the above purity grades are disclosed herein as if each individual combination was specifically and explicitly recited.

In particular embodiments, the MTC is a high purity MTC wherein ‘high purity’ is characterised by a purity of greater than 98% and one or more of the following:
(i) less than 2% Azure B as impurity;
(ii) less than 0.13% MVB (Methylene Violet Bernstein) as impurity; or
(iii) an elementals purity better than the European Pharmacopeia limits of less than 100 μg/g Aluminium (AI); less than 1 μg/g Cadmium (Cd); less than 100 μg/g Chromium (Cr); less than 300 μg/g Copper (Cu); less than 10 μg/g Tin (Sn); less than 200 μg/g Iron (Fe); less than 10 μg/g Manganese (Mn); less than 1 μg/g Mercury (Hg); less than 10 μg/g Molybdenum (Mo); less than 10 μg/g Nickel (Ni); less than 10 μg/g Lead (Pb); and less than 100 μg/g Zinc (Zn).

In particular embodiments, the MTC is a high purity MTC wherein high-purity is characterised by a purity of greater than 98% and one or more of the following: (i) less than 1% Azure B as impurity;
(ii) less than 0.15% Azure A as impurity;
(iii) less than 0.15% Azure C as impurity;
(iv) less than 0.13% Methylene Violet Bernthsen (MVB) as impurity;
(v) an elementals purity better than the European Pharmacopeia limits of less than 100 μg/g Aluminium (AI); less than 1 μg/g Cadmium (Cd); less than 100 μg/g Chromium (Cr); less than 300 μg/g Copper (Cu); less than 10 μg/g Tin (Sn); less than 200 μg/g Iron (Fe); less than 10 μg/g Manganese (Mn); less than 1 μg/g Mercury (Hg); less than 10 μg/g Molybdenum (Mo); less than 10 μg/g Nickel (Ni); less than 10 μg/g Lead (Pb); and less than 100 μg/g Zinc (Zn).

In particular embodiments, the MTC is a high purity MTC wherein high-purity is characterised by a purity of greater than 98% and one or more of the following:
(i) less than 1% Azure B as impurity;
(ii) less than 0.15% Azure A as impurity;
(iii) less than 0.15% Azure C as impurity;
(iv) less than 0.05% Methylene Violet Bernthsen (MVB) as impurity; or
(v) an elementals purity better than the European Pharmacopeia limits of less than 100 μg/g Aluminium (AI); less than 1 μg/g Cadmium (Cd); less than 100 μg/g Chromium (Cr); less than 300 μg/g Copper (Cu); less than 10 μg/g Tin (Sn); less than 200 μg/g Iron (Fe); less than 10 μg/g Manganese (Mn); less than 1 μg/g Mercury (Hg); less than 10 μg/g Molybdenum (Mo); less than 10 μg/g Nickel (Ni); less than 10 μg/g Lead (Pb); and less than 100 μg/g Zinc (Zn).

In particular embodiments, the MTC is a high purity MTC wherein high-purity is characterised by at least 98% purity and less than 1% Azure B as impurity.

In particular embodiments, the MTC is a high purity MTC wherein high-purity is characterised by:
(i) at least 98% purity
(i) less than 1% Azure B as impurity; and
(ii) an elementals purity better than the European Pharmacopeia limits of less than 100 μg/g Aluminium (AI); less than 1 μg/g Cadmium (Cd); less than 100 μg/g Chromium (Cr); less than 300 μg/g Copper (Cu); less than 10 μg/g Tin (Sn); less than 200 μg/g Iron (Fe); less than 10 μg/g Manganese (Mn); less than 1 μg/g Mercury (Hg); less than 10 μg/g Molybdenum (Mo); less than 10 μg/g Nickel (Ni); less than 10 μg/g Lead (Pb); and less than 100 μg/g Zinc (Zn).

In particular embodiments, the MTC is a high purity MTC wherein high-purity is characterised by at least 98% purity and an elementals purity better than the European Pharmacopeia limits of less than 100 μg/g Aluminium (AI); less than 1 μg/g Cadmium (Cd); less than 100 μg/g Chromium (Cr); less than 300 μg/g Copper (Cu); less than 10 μg/g Tin (Sn); less than 200 μg/g Iron (Fe); less than 10 μg/g Manganese (Mn); less than 1 μg/g Mercury (Hg); less than 10 μg/g Molybdenum (Mo); less than 10 μg/g Nickel (Ni); less than 10 μg/g Lead (Pb); and less than 100 μg/g Zinc (Zn).

Methods for the production of ‘high purity’ diaminophenothiazinium compounds, including MTC, are described, for example, in WO2006/032879 and WO2008/007074 (WisTa Laboratories Ltd) and in WO2008/006979 (Provence Technologies).

A preferred MTC polymorph for use in the methods and compositions described herein is ‘form A’ described in WO2011/036561 which is a pentahydrate, at a “high purity” described above. That has a molecular weight of around 409.9. Based on a molecular weight of 284.1 for the MT⁺ core, the weight factor for using this MT compound in the invention is 1.44.

Other weight factors can be calculated for example MT compounds herein, and the corresponding dosage ranges can be calculated therefrom.

Other example MT compounds are described in WO2007/110630. Their molecular weight (anhydrous) and weight factor is also shown:

| | | | MT⁺ anhydrous |
|---|---|---|---|
| | Compound | Molecular weight | weight factor |
| 10 | MTC•0.5ZnCl₂ | 388.0 | 1.36 |
| 11 | MTI | 411.3 | 1.45 |
| 12 | MTI•HI | 539.2 | 2.73 |
| 13 | MT•NO₃ | 346.4 | 1.22 |

The dosages described herein with respect to MT thus apply mutatis mutandis for these MT containing compounds, as adjusted for their molecular weight, and for choice of hydrate if used. For example MTC·0.5ZnCl₂ (also referred to as ‘METHYLENE BLUE ZINC CHLORIDE DOUBLE SALT; CI 52015) may be obtained commercially as a monohydrate by several suppliers, which would have a molecular weight higher by 18, and correspondingly altered weight factor. MTI is reportedly available as a hemihydrate.

Any of the MT compounds described herein, may be formulated with a reducing agent. In particular they may be formulated with a reducing agent such as ascorbate or other another food grade antioxidant.

In the various aspects of the invention described herein (as they relate to an MT-containing compound) the MT-containing compound may optionally be any of those compounds described above:

In one embodiment, it is compound 1.

In one embodiment, it is compound 2.

In one embodiment, it is compound 3.

In one embodiment, it is compound 4.

In one embodiment, it is compound 5.

In one embodiment, it is compound 6.

In one embodiment, it is compound 7.

In one embodiment, it is compound 8.

In one embodiment, it is compound 9.

In one embodiment, it is compound 10.

In one embodiment, it is compound 11.

In one embodiment, it is compound 12.

In one embodiment, it is compound 13.

Although the MT containing compounds described herein are themselves salts, they may also be provided in the form of a mixed salt (i.e., the compound of the invention in combination with another salt). Such mixed salts are intended to be encompassed by the term “and pharmaceutically acceptable salts thereof”. Unless otherwise specified, a reference to a particular compound also includes salts thereof.

The compounds of the invention may also be provided in the form of a solvate or hydrate. The term “solvate” is used herein in the conventional sense to refer to a complex of solute (e.g., compound, salt of compound) and solvent. If the solvent is water, the solvate may be conveniently referred to as a hydrate, for example, a mono-hydrate, a di-hydrate, a tri-hydrate, a penta-hydrate etc. Unless otherwise specified, any reference to a compound also includes solvate and any hydrate forms thereof.

MT compounds for use in the present invention may include mixtures of the oxidised and reduced form.

In particular, the LMT-containing compounds may include oxidised (MT⁺) compounds as ‘impurities’ during synthesis, and may also oxidize (e.g., autoxidize) after synthesis to give the corresponding oxidized forms. Thus, it is likely, if not inevitable, that compositions comprising the compounds of the present invention will contain, as an impurity, at least some of the corresponding oxidized compound. For example an “LMT” salt may include 10 to 15% of MT⁺ salt.

When using mixed MT compounds the MT dose can be readily calculated using the molecular weight factors of the compounds present.

In various embodiments, the DAPTZ compound is selected from methylthioninium chloride (MTC); leuco-methylthioninium (bis)mesylate (LMTM); and a combination thereof.

In various embodiments, the DAPTZ compound as the active ingredient may comprise methylthioninium chloride (MTC).

In various embodiments, the DAPTZ compound as the active ingredient may comprise leuco-methylthioninium (bis)mesylate (LMTM).

In various embodiments, the DAPTZ compound as the active ingredient may comprises a combination of different DAPTZ compounds.

In various embodiments, the DAPTZ compound as the active ingredient may comprise methylthioninium chloride (MTC) and leuco-methylthioninium (bis)mesylate (LMTM) in combination.

In various embodiments, the effective amount of DAPTZ compound comprises 3,7-bis(dimethylamino) phenothiazin-5-ium chloride.

The “MT compound”, although present in relatively low amount, is the active agent of the dosage unit, which is to say is intended to have the therapeutic or prophylactic effect in respect of a neurodegenerative disorder of protein aggregation. Rather, the other ingredients in the dosage unit will be therapeutically inactive e.g. carriers, diluents, or excipients. Thus, preferably, there will be no other active ingredient in the dosage unit, no other agent intended to have a therapeutic or prophylactic effect in respect of a disorder for which the dosage unit is intended to be used.

However, in various embodiments, the formulation may further comprise other accompanying active ingredients, rapid release elements, additives, excipients, diluents, binders, lubricants, disintegrators, fillers, stabilizers, surfactants, antioxidants, or combinations thereof. In various embodiments, the other accompanying active ingredients may comprise any known antibiotics, anti-inflammatories antifungals, or antivirals.

According to various embodiments, there is an oral formulation comprising a DAPTZ compound and gastro-retentive platform for use in treatment and/or prevention of a disease. In various embodiments, the oral formulation comprises any formulation described herein above.

In as further aspect there is provided a method of treatment or prophylaxis of a disease in a subject, which method comprises orally (or via a nasogastric tube) administering to said subject a composition of the invention. Such methods may comprise administering an effective amount of the diaminophenothiazine compound in the formulation with the gastro-retentive platform to a human suffering from the disease. Preferably the diaminophenothiazine compound is selected from methylthioninium chloride (MTC); leuco-methylthioninium (bis)mesylate (LMTM); and a combination thereof. In some embodiments the effective amount of the diaminophenothiazine compound in the formulation with the gastro-retentive platform is administered daily.

In one embodiment a formulation (e.g. oral formulation) described herein may be used in treatment and/or prevention of gastric perforations or ulcerations of an upper gastrointestinal tract caused by infective agents. In another embodiment there is provided use of a composition of the invention in the manufacture of a formulation for the treatment and/or prevention of gastric perforations or ulcerations of an upper gastrointestinal tract caused by infective agents.

In other embodiments described hereinafter the disease is a neurodegenerative disorder of protein aggregation.

Thus one aspect of the invention is the use of a gastro-retained composition as described herein, to regulate (e.g., to reverse and/or inhibit) the aggregation of a protein, for example, aggregation of a protein associated with a neurodegenerative disease and/or clinical dementia. The aggregation will be associated with a disease state as discussed below.

Similarly, one aspect of the invention pertains to a method of regulating (e.g., reversing and/or inhibiting) the aggregation of a protein in the brain of a mammal, which aggregation is associated with a disease state as described herein, the treatment comprising the step of administering to said mammal in need of said treatment, a prophylactically or therapeutically effective amount of an MT gastro-retained composition as described herein, that is an inhibitor of said aggregation.

Disease conditions treatable via the present invention are discussed in more detail below.

### Methods of Treatment

Another aspect of the present invention, as explained above, pertains to a method of treatment comprising administering to a patient in need of treatment a prophylactically or therapeutically effective amount of a compound as described herein, preferably in the form of a pharmaceutical composition.

### Use in Methods of Therapy

Another aspect of the present invention pertains to a gastro-retained composition as described herein, for use in a method of treatment (e.g., of a disease condition) of the human or animal body by therapy.

### Use in the Manufacture of Medicaments

Another aspect of the present invention pertains to use of an MT gastro-retained composition as described herein, in the manufacture of a medicament for use in treatment (e.g., of a disease condition).

### Diseases of Protein Aggregation

The compositions of the present invention are useful in the treatment or prophylaxis of diseases of protein aggregation.

Thus, in some embodiments, the disease condition is a disease of protein aggregation, and, for example, the treatment is with an amount of a gastro-retained composition as described herein, sufficient to inhibit the aggregation of the protein associated with said disease condition.

The following Table lists various disease-associated aggregating proteins and the corresponding neurodegenerative disease of protein aggregation. The use of the compounds and compositions of the invention in respect of these proteins or diseases is encompassed by the present invention.

Diseases of protein aggregation

| | | | | |
|---|---|---|---|---|
| | | | Fibril | |
| | | Aggregating | subunit | |
| | | domain and/or | size | |
| Protein | Disease | mutations | (kDa) | Reference |

Neurodegenerative disorders

| | | | | |
|---|---|---|---|---|
| Prion | Prion diseases | Inherited and | 27 | Prusiner (1998) |
| protein | | sporadic forms | | |
| | (CJD, nvCJD, Fatal | PrP-27-30; many | 27 | Prusiner (1998) |
| | familial insomnia, | mutations. | | |
| | Gerstmann-Straussler- | | | |
| | Scheinker syndrome, | | | |
| | Kuru) | | | |
| | | Fibrillogenic | | Gasset et al. (1992) |
| | | domains: 113- | | |
| | | 120, 178-191, | | |
| | | 202-218. | | |
| Tau protein | Alzheimer's disease, | Inherited and | 10-12 | Wischik et al. (1988) |
| | Down's syndrome, FTDP- | sporadic forms | | |
| | 17, CBD, post-encephalitic | | | |
| | parkinsonism, Pick's | | | |
| | disease, parkinsonism | | | |
| | with dementia complex of | | | |
| | Guam | | | |
| | | Truncated tau | 10-12 | Wischik et al. (1988) |
| | | (tubulin-binding | | |
| | | domain) 297-391. | | |
| | | Mutations in tau | | Hutton et al. (1998) |
| | | in FTDP-17. | | |
| | | Many mutations | | Czech et al. (2000) |
| | | in presenilin | | |
| | | proteins. | | |
| Amyloid | Alzheimer's disease, | Inherited and | 4 | Glenner & Wong, |
| β-protein | Down's syndrome | sporadic forms | | (1984) |
| | | Amyloid β- | 4 | Glenner & Wong, |
| | | protein; 1-42(3). | | (1984) |
| | | Mutations in APP | | Goate et al. (1991) |
| | | in rare families. | | |
| Huntingtin | Huntington's disease | N-termini of | 40 | DiFiglia et al. (1997) |
| | | protein with | | |
| | | expanded | | |
| | | glutamine | | |
| | | repeats. | | |
| Ataxin) | Spinocerebellar ataxias | Proteins with | | Paulson et al. (1999) |
| | (SCA1, 2, 3, 7) | expanded | | |
| | | glutamine | | |
| | | repeats. | | |
| Atrophin | Dentatorubropallidoluysian | Proteins with | | Paulson et al. (1999) |
| | atrophy (DRPLA) | expanded | | |
| | | glutamine | | |
| | | repeats. | | |
| Androgen | Spinal and bulbar | Proteins with | | Paulson et al. (1999) |
| receptor | muscular atrophy | expanded | | |
| | | glutamine | | |
| | | repeats. | | |
| Neuroserpin | Familial encephalopathy | Neuroserpin; | 57 | Davis et al. (1999) |
| | with neuronal inclusion | S49P, S52R. | | |
| | bodies (FENIB) | | | |
| α-Synuclein | Parkinson's disease, | Inherited and | 19 | Spillantini et al. |
| | dementia with Lewy | sporadic forms | | (1998) also |
| | bodies, multiple system | | | PCT/GB2007/001105 |
| | atrophy | | | |
| | | A53T, A30P in | | Polymeropoulos et |
| | | rare autosomal- | | al. (1997) |
| | | dominant PD | | |
| | | families. | | |
| TDP-43 | FTLD-TDP | Several TDP-43 | 10-43 | Mackenzie et al. |
| | Amyotrophic lateral | mutations | 10-43 | (2010) |
| | sclerosis | Several TDP-43 | | Mackenzie et al. |
| | | mutations | | (2010) |
| Cystatin C | Hereditary cerebral | Cystatin C less | 12-13 | Abrahamson et al. |
| | angiopathy (Icelandic) | 10 residues; | | (1992) |
| | | L68Q. | | |
| Superoxide | Amyotrophic lateral | SOD1 mutations. | 16 | Shibata et al. (1996) |
| dismutase 1 | sclerosis | | | |

As described in WO 02/055720, WO2007/110630, and WO2007/110627, diaminophenothiazines have utility in the inhibition of such protein aggregating diseases.

Thus it will be appreciated that, except where context requires otherwise, description of embodiments with respect to tau protein or tau-like proteins (e.g., MAP2; see below), should be taken as applying equally to the other proteins discussed herein (e.g., β-amyloid, synuclein, prion, etc.) or other proteins which may initiate or undergo a similar pathological aggregation by virtue of conformational change in a domain critical for propagation of the aggregation, or which imparts proteolytic stability to the aggregate thus formed (see, e.g., the article by Wischik et al. in “Neurobiology of Alzheimer's Disease”, 2nd Edition, 2000, Eds. Dawbarn, D. and Allen, S. J., The Molecular and Cellular Neurobiology Series, Bios Scientific Publishers, Oxford). All such proteins may be referred to herein as “aggregating disease proteins.”

Likewise, where mention is made herein of “tau-tau aggregation”, or the like, this may also be taken to be applicable to other “aggregating-protein aggregation”, such as β-amyloid aggregation, prion aggregation, synuclein aggregation, etc. The same applies for “tau proteolytic degradation” etc.

### Preferred Aggregating Disease Target Proteins

Preferred embodiments of the invention are based on tau protein. The term “tau protein,” as used herein, refers generally to any protein of the tau protein family. Tau proteins are characterised as being one among a larger number of protein families which co-purify with microtubules during repeated cycles of assembly and disassembly (see, e.g., Shelanski et al., 1973, Proc. Natl. Acad. Sci. USA, Vol. 70, pp. 765-768), and are known as microtubule-associated-proteins (MAPs). Members of the tau family share the common features of having a characteristic N-terminal segment, sequences of approximately 50 amino acids inserted in the N-terminal segment, which are developmentally regulated in the brain, a characteristic tandem repeat region consisting of 3 or 4 tandem repeats of 31-32 amino acids, and a C-terminal tail.

MAP2 is the predominant microtubule-associated protein in the somatodendritic compartment (see, e.g., Matus, A., in “Microtubules” [Hyams and Lloyd, Eds.] pp. 155-166, John Wiley and Sons, New York, USA). MAP2 isoforms are almost identical to tau protein in the tandem repeat region, but differ substantially both in the sequence and extent of the N-terminal domain (see, e.g., Kindler and Garner, 1994, Mol. Brain Res., Vol. 26, pp. 218-224). Nevertheless, aggregation in the tandem-repeat region is not selective for the tau repeat domain. Thus it will be appreciated that any discussion herein in relation to tau protein or tau-tau aggregation should be taken as relating also to tau-MAP2 aggregation, MAP2-MAP2 aggregation, and so on.

In some embodiments, the protein is tau protein.

In some embodiments, the protein is a synuclein, e.g., α- or β-synuclein.

In some embodiments, the protein is TDP-43.

TAR DNA-Binding Protein 43 (TDP-43) is a 414 amino acid protein encoded by TARDBP on chromosome 1p36.2. The protein is highly conserved, widely expressed, and predominantly localised to the nucleus but can shuttle between the nucleus and cytoplasm (Mackenzie et al 2010). It is involved in transcription and splicing regulation and may have roles in other processes, such as: microRNA processing, apoptosis, cell division, stabilisation of messenger RNA, regulation of neuronal plasticity and maintenance of dendritic integrity. Furthermore, since 2006 a substantial body of evidence has accumulated in support of the TDP-43 toxic gain of function hypothesis in amyotrophic lateral sclerosis (ALS). TDP-43 is an inherently aggregation-prone protein and aggregates formed in vitro are ultrastructurally similar to the TDP-43 deposits seen in degenerating neurones in ALS patients (Johnson et al 2009). Johnson et al (2008) showed that when TDP-43 is overexpressed in a yeast model only the aggregated form is toxic. Several in vitro studies have also shown that C-terminal fragments of TDP-43 are more likely than full-length TDP-43 to form insoluble cytoplasmic aggregates that become ubiquitinated, and toxic to cells (Arai et al 2010; Igaz et al 2009; Nonaka et al 2009; Zhang et al 2009). Though Nonaka et al (2009) suggested that these cytoplasmic aggregates bind the endogenous full-length protein depleting it from the nucleus, Zhang et al (2009) found retention of normal nuclear expression, suggesting a purely toxic effect for the aggregates. Yang et al (2010) have described the capture of full-length TDP-43 within aggregates of C- and N-terminal fragments of TDP-43 in NSC34 motor neurons in culture. Neurite outgrowth, impaired as a result of the presence of such truncated fragments, could be rescued by overexpression of the full-length protein. Although the role of neurite outgrowth in vivo has not been established, this model would support the suggestion made by Nonaka and colleagues for a role of TDP-43 aggregation in ALS pathogenesis.

Mutant TDP-43 expression in cell cultures has repeatedly been reported to result in increased generation of C-terminal fragments, with even greater cytoplasmic aggregation and toxic effects than the wild-type protein (Kabashi et al 2008; Sreedharan et al 2008; Johnson et al 2009; Nonaka et al 2009; Arai et al 2010; Barmarda et al 2010; Kabashi et al 2010).

Where the protein is tau protein, in some embodiments of the present invention, there is provided a method of inhibiting production of protein aggregates (e.g. in the form of paired helical filaments (PHFs), optionally in neurofibrillary tangles (NFTs) in the brain of a mammal, the treatment being as described above.

### Preferred Indications—Diseases of Protein Aggregation

In one embodiment the present invention is used for the treatment of Alzheimer's disease (AD)—for example mild, moderate or severe AD.

Notably it is not only Alzheimer's disease (AD) in which tau protein (and aberrant function or processing thereof) may play a role. The pathogenesis of neurodegenerative disorders such as Pick's disease and progressive supranuclear palsy (PSP) appears to correlate with an accumulation of pathological truncated tau aggregates in the dentate gyrus and stellate pyramidal cells of the neocortex, respectively. Other dementias include fronto-temporal dementia (FTD); FTD with parkinsonism linked to chromosome 17 (FTDP-17); disinhibition-dementia-parkinsonism-amyotrophy complex (DDPAC); pallido-ponto-nigral degeneration (PPND); Guam-ALS syndrome; pallido-nigro-luysian degeneration (PNLD); cortico-basal degeneration (CBD) and others (see, e.g., the article by Wischik et al. in “Neurobiology of Alzheimer's Disease”, 2nd Edition, 2000, Eds. Dawbarn, D. and Allen, S. J., The Molecular and Cellular Neurobiology Series, Bios Scientific Publishers, Oxford; especially Table 5.1). All of these diseases, which are characterized primarily or partially by abnormal tau aggregation, are referred to herein as “tauopathies”.

Thus, in some embodiments, the disease condition is a tauopathy.

In some embodiments, the disease condition is a neurodegenerative tauopathy.

In some embodiments, the disease condition is selected from Alzheimer's disease (AD), Pick's disease, progressive supranuclear palsy (PSP), fronto temporal dementia (FTD), FTD with parkinsonism linked to chromosome 17 (FTDP 17), frontotemporal lobar degeneration (FTLD) syndromes; disinhibition-dementia-parkinsonism-amyotrophy complex (DDPAC), pallido-ponto-nigral degeneration (PPND), Guam-ALS syndrome, pallido nigro luysian degeneration (PNLD), cortico-basal degeneration (CBD), dementia with argyrophilic grains (AgD), dementia pugilistica (DP) or chronic traumatic encephalopathy (CTE), Down's syndrome (DS), dementia with Lewy bodies (DLB), subacute sclerosing panencephalitis (SSPE), MCI, Niemann-Pick disease, type C (NPC), Sanfilippo syndrome type B (mucopolysaccharidosis III B), or myotonic dystrophies (DM), DM1 or DM2, or chronic traumatic encephalopathy (CTE).

In some embodiments, the disease condition is a lysosomal storage disorder with tau pathology. NPC is caused by mutations in the gene NPC1, which affects cholesterol metabolism (Love et al 1995) and Sanfilippo syndrome type B is caused by a mutation in the gene NAGLU, in which there is lysosomal accumulation of heparin sulphate (Ohmi et al. 2009). In these lysosomal storage disorders, tau pathology is observed and its treatment may decrease the progression of the disease. Other lysosomal storage disorders may also be characterised by accumulation of tau.

Use of DAPTZ salts in the treatment of Parkinson's disease and MCI is described in more detail in PCT/GB2007/001105 and PCT/GB2008/002066.

In some embodiments, the disease condition is Parkinson's disease, MCI, or Alzheimer's disease.

In some embodiments, the disease condition is MCI or Alzheimer's disease.

In some embodiments, the disease condition is MCI.

Optionally, treating MCI according to methods of the invention comprises inhibiting decline, preventing an expected decline, or improving the condition. For example, in some embodiments, treatment of MCI may comprise improving cognitive ability or function in a subject.

In some embodiments, the disease condition is Huntington's disease or other polyglutamine disorder such as spinal bulbar muscular atrophy (or Kennedy disease), and dentatorubropallidoluysian atrophy and various spinocerebellar ataxias.

In some embodiments, the disease condition is an FTLD syndrome (which may for example be a tauopathy or TDP-43 proteinopathy, see below).

In some embodiments, the disease condition is PSP or ALS.

TDP-43 proteinopathies include amyotrophic lateral sclerosis (ALS; ALS-TDP) and frontotemporal lobar degeneration (FTLD-TDP).

The role of TDP-43 in neurodegeneration in ALS and other neurodegenerative disorders has been reviewed in several recent publications (Chen-Plotkin et al 2010; Gendron et al 2010; Geser et al 2010; Mackenzie et al 2010).

ALS is a neurodegenerative disease, characterised by progressive paralysis and muscle wasting, consequent on the degeneration of both upper and lower motor neurones in the primary motor cortex, brainstem and spinal cord. It is sometimes referred to as motor neuron disease (MND) but there are diseases other than ALS which affect either upper or lower motor neurons. A definite diagnosis requires both upper and lower motor neurone signs in the bulbar, arm and leg musculature with clear evidence of clinical progression that cannot be explained by any other disease process (Wijesekera and Leigh 2009).

Although the majority of cases are ALS-TDP, there are other cases where the pathological protein differs from TDP-43. Misfolded SOD1 is the pathological protein in ubiquitin-positive inclusions in ALS with SOD1 mutations (Seetharaman et al 2009) and in a very small subset (approximately 3-4%) of familial ALS, due to mutations in FUS (fused in sarcoma protein), the ubiquitinated pathological protein is FUS (Vance et al 2009; Blair et al 2010). FUS, like TDP-43, appears to be important in nuclear-cytoplasmic shuttling although the ways in which impaired nuclear import of FUS remains unclear. A new molecular classification of ALS, adapted from Mackenzie et al (2010), reflects the distinct underlying pathological mechanisms in the different subtypes (see Table below).

New Molecular Classification of ALS (modified from Mackenzie et al 2010). In the majority of cases, TDP-43 is the pathological ubiquitinated protein found in ALS.

Ubiquitin-positive inclusions in ALS

Ubiquitinated disease protein

| | TDP-43 | FUS | SOD1 |
|---|---|---|---|
| Clinico- | ALS-TDP | ALS-FUS | ALS-SOD1 |
| pathologic | | | |
| subtype | | | |
| Associated | TARDBP | FUS | SOD1 |
| genotype | | | |
| Frequency of ALS | Common | Rare | Rare |
| cases | | | |

Amyotrophic lateral sclerosis has been recognised as a nosological entity for almost a century and a half and it is recognised in ICD-10 is classified as a subtype of MND in ICD 10 (G12.2). Reliable clinical diagnostic criteria are available for ALS, which differ little from Charcot's original description, and neuropathological criteria, reflecting the underlying molecular pathology, have also been agreed.

While ALS is classified pathologically into three subgroups, ALS-TDP, ALS-SOD1 and ALS-FUS, both latter conditions are rare. The largest study to date showed all sporadic ALS cases to have TDP-43 pathology (Mackenzie et al 2007). Only around 5% of ALS is familial (Byrne et al 2010) and mutations in SOD1, the commonest mutations found in FALS, account for between 12-23% of cases (Andersen et al 2006). SOD1 may also be implicated in 2-7% of SALS. Mutations in FUS appear to be far less common, accounting for only around 3-4% of FALS (Blair et al 2010). So it can be reliably predicted that a clinical case of SALS will have TDP-43 based pathology. Similarly this can be reliably predicted in FALS due to mutations in TDP-43, which account for around 4% of cases (Mackenzie et al 2010). ALS with mutations in: VCP, accounting for 1-2% of FALS (Johnson et al 2010), ANG (Seilhean et al 2009), and CHMP2B (Cox et al 2010) have also been reported to be associated with TDP-43 positive pathology. Although SOD1, FUS and ATXN2 mutations have not been found to be associated with TDP-43 positive aggregates, it has however been reported that TDP-43 is implicated in the pathological processes putatively arising from these mutations (Higashi et al 2010; Ling et al 2010; Elden et al 2010).

It is therefore established that TDP-43 has an important, and potentially central role, in the pathogenesis of the vast majority of SALS cases and may be implicated in the pathogenesis of a significant proportion of FALS. ALS is now widely considered to be a TDP-43 proteinopathy (Neumann et al 2009) and numerous in vitro, and in vivo studies provide support to the hypothesis that toxic gain of function, due to TDP-43 aggregation is responsible for at least some of the neurotoxicity in the disease.

FTLD syndromes are insidious onset, inexorably progressive, neurodegenerative conditions, with peak onset in late middle age. There is often a positive family history of similar disorders in a first degree relative.

Behavioural variant FTD is characterised by early prominent change in social and interpersonal function, often accompanied by repetitive behaviours and changes in eating pattern. In semantic dementia there are prominent word finding problems, despite otherwise fluent speech, with degraded object knowledge and impaired single word comprehension on cognitive assessment. Progressive non-fluent aphasia presents with a combination of motor speech problems and grammatical deficits. The core clinical diagnostic features for these three FTLD syndromes are shown in the Table below and the full criteria in Neary et al (1998).

### Clinical Profile and Core Diagnostic Features of FTLD Syndromes

| FTLD Syndrome -Clinical Profile | Core Diagnostic Features |
|---|---|
| Frontotemporal Dementia | 1. Insidious onset and gradual |
| Character change and disordered social | progression |
| conduct are the dominant features initially | 2. Early decline in social interpersonal |
| and throughout the disease course. | conduct |
| Instrumental functions of perception, | 3. Early impairment in regulation of |
| spatial skills, praxis and memory are intact | personal conduct |
| or relatively well preserved. | 4. Early emotional blunting |
| | 5. Early loss of insight |
| Semantic Dementia | A) Insidious onset and gradual progression |
| Semantic disorder (impaired | B) Language disorder characterised by |
| understanding of word meaning and/or | 1. Progressive, fluent empty speech |
| object identity) is the dominant feature | 2. Loss of word meaning manifest by |
| initially and throughout the disease course. | impaired naming and comprehension |
| Other aspects of cognition, including | 3. Semantic paraphasias and/or |
| autobiographic memory, are intact or | 4. Perceptual disorder characterised by |
| relatively well preserved. | 1. Prosopagnosia: impaired |
| | recognition of identity of familiar faces |
| | and/or |
| | 2. Associative agnosia: impaired |
| | recognition of object identity |
| | C) Preserved perceptual matching and |
| | drawing reproduction |
| | D) Preserved single word repetition |
| | E) Preserved ability to read aloud and |
| | write to dictation orthographically regular |
| | words |
| Progressive Non-fluent Aphasia | A) Insidious onset and gradual |
| Disorder of expressive language is the | progression |
| dominant feature initially and throughout | B) Non-fluent spontaneous speech with at |
| the disease course. Other aspects of | least one of the following: agrammatism, |
| cognition are intact or relatively well | phonemic paraphasias or anomia |
| preserved. | |

The discovery that TDP-43-positive inclusions characterize ALS and FTLD-TDP (Neumann et al 2006) was quickly followed by the identification of missense mutations in the TARDBP gene in both familial and sporadic cases of ALS (Gitcho et al 2008; Sreedharan et al., 2008). So far, 38 different TARDBP mutations have been reported in 79 genealogically unrelated families worldwide (Mackenzie et al 2010). TARDBP mutations account for approximately 4% of all familial and around 1.5% of sporadic ALS cases.

As of December 2010, mutations in thirteen genes which are associated with familial and sporadic ALS have been identified. Linkage of ALS to five other chromosome loci has been demonstrated but thus far specific mutations have not been identified.

### TDP-43 Proteinopathies

MT has a mode of action which targets and can reduce TDP-43 protein aggregation in cells, which is a pathological feature of the vast majority of both familial and sporadic ALS and is also characteristic of FTLD-P.

In addition laboratory data shows that methylthioninium inhibits the formation of TDP-43 aggregates in SH-SY5Y cells. Following treatment with 0.05 μM MT, the number of TDP-43 aggregates was reduced by 50%. These findings were confirmed by immunoblot analysis (Yamashita et al 2009).

The compounds and compositions of the invention may therefore be useful for the treatment of amyotrophic lateral sclerosis (ALS) and frontotemporal lobar degeneration (FTLD).

### Huntington's Disease and Polyglutamine Disorders

MT can reduce polyglutamine protein aggregation in cells, which is a pathological feature of Huntington's disease. Huntington's disease is caused by expansion of a translated CAG repeat located in the N-terminus of huntingtin. Wild-type chromosomes contain 6-34 repeats whereas, in Huntington's disease, chromosomes contain 36-121 repeats. The age of onset of disease correlates inversely with the length of the CAG tracts that code for polyglutamine repeats within the protein.

Laboratory data shows that methylthioninium inhibits the formation of aggregates of a huntingtin derivative containing a polyglutamine stretch of 102 residues in zebrafish (van Bebber et al. 2010). MT, when tested at 0, 10 and 100 μM, prevented the formation of such aggregates in zebrafish in a dose dependent manner.

The compounds and compositions of the invention may therefore be useful for the treatment of Huntington's disease and other polyglutamine disorders such as spinal bulbar muscular atrophy (or Kennedy disease), and dentatorubropallidoluysian atrophy and various spinocerebellar ataxias (Orr & Zoghbi, 2007).

### Mitochondrial Diseases and Lafora Disease

The organ most frequently affected in mitochondrial disorders, particularly respiratory chain diseases (RCDs), in addition to the skeletal muscle, is the central nervous system (CNS). CNS manifestations of RCDs comprise stroke-like episodes, epilepsy, migraine, ataxia, spasticity, movement disorders, psychiatric disorders, cognitive decline, or even dementia (mitochondrial dementia). So far mitochondrial dementia has been reported in MELAS, MERRF, LHON, CPEO, KSS, MNGIE, NARP, Leigh syndrome, and Alpers-Huttenlocher disease (Finsterer, 2009). There are four complexes in the mitochondrial respiration chain, involving a series of electron transfers. Abnormal function of any of these complexes can result in mitochondrial diseases secondary to an abnormal electron transport chain and subsequent abnormal mitochondrial respiration. Complex III of the mitochondrial respiration chain acts to transfer electrons to cytochrome c.

Compounds and compositions of the invention may also be used to treat mitochondrial diseases which are associated with a deficient and/or impaired complex III function of the respiration chain. The compounds have the ability to act as effective electron carrier and/or transfer, as the thioninium moiety has a low redox potential converting between the oxidised and reduced form. In the event of an impaired and/or deficient function of Complex III leading to mitochondrial diseases, compounds of the invention are also able to perform the electron transportation and transfer role of complex III because of the ability of the thioninium moiety to shuttle between the oxidised and reduced form, thus acting as an electron carrier in place of sub-optimally functioning complex III, transferring electrons to cytochrome c.

Compounds and compositions of the invention also have the ability to generate an active thioninium moiety that has the ability to divert misfolded protein/amino acid monomers/oligomers away from the Hsp70 ADP-associated protein accumulation and/or refolding pathways, and instead rechannel these abnormal folded protein monomers/oligomers to the pathway that leads directly to the Hsp70 ATP-dependent ubiquitin-proteasome system (UPS), a pathway which removes these misfolded proteins/amino acid monomers/oligomers via the direct route (Jinwal et al. 2009).

Lafora disease (LD) is an autosomal recessive teenage-onset fatal epilepsy associated with a gradual accumulation of poorly branched and insoluble glycogen, termed polyglucosan, in many tissues. In the brain, polyglucosan bodies, or Lafora bodies, form in neurons. Inhibition of Hsp70 ATPase by MT (Jinwal et al. 2009) may upregulate the removal of misfolded proteins. Lafora disease is primarily due to a lysosomal ubiquitin-proteasomal system (UPS) defect because of a mutation in either the Laforin or Malin genes, both located on Chromosome 6, which result in inclusions that may accelerate the aggregation of misfolded tau protein. Secondary mitochondrial damage from the impaired UPS may further result in a suppressed mitochondrial activity and impaired electron transport chain leading to further lipofuscin and initiating the seizures that are characteristic of Lafora disease.

The MT moiety may disaggregate existing tau aggregates, reduce more tau accumulating and enhance lysosomal efficiency by inhibiting Hsp70 ATPase. MT may lead to a reduction in tau tangles by enhancing the ubiquitin proteasomal system removal of tau monomers/oligomers, through its inhibitory action on Hsp70 ATPase.

Thus compounds and compositions of the present invention may have utility in the treatment of Lafora disease.

In various embodiments, the formulation (e.g. oral formulation) comprising a DAPTZ compound and gastro-retentive platform may be for use in treatment and/or prevention of diseases caused by infective agents such as bacteria, fungi or viruses that effect the stomach or upper gastrointestinal tract.

In various embodiments the formulation (e.g. oral formulation) comprising a DAPTZ compound and gastro-retentive platform may be for use in treatment and/or prevention of gastric perforations and ulcerations.

In various embodiments the disease is gastric perforations and/or ulcerations.

In various embodiments, the formulation is prepared to be administered daily. In various embodiments, the formulation is prepared to be administered weekly. In various embodiments, the formulation for use in treatment of a disease is prepared to be administered daily. In various embodiments, the formulation for use in prevention of a disease is prepared to be administered weekly.

In various embodiments, the effective amount of DAPTZ compound with 2 to 3 times an amount of gastro-retentive platform is formulated for use as the oral formulation for administration.

In various embodiments, the effective amount of DAPTZ compound in an oral formulation with gastro-retentive platform is administered daily. In various embodiments, the oral formulation comprises any formulation described herein above.

In various embodiments, less than 5 μg of DAPTZ compound is administered daily per kilogram of the weight of the human subject.

In various embodiments, less than 4.5 μg of DAPTZ compound is administered daily per kilogram of the weight of the human subject.

In various embodiments, less than 4 μg of DAPTZ compound is administered daily per kilogram of the weight of the human subject.

The dosages used in the methods described herein will depend on the DAPTZ compound and the disease in question. Nevertheless it is believed that typically the average MT daily dose may usefully be from around any of 0.1, 0.5, 1, 2, 2.5, 3, 3.5, 4 mg to around any of 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59 or 60 mg.

Further example dosages are 8 or 16 or 24 mg/day.

Wherein the pharmaceutical composition is the rapid release solid tablet, a total daily dose is preferably between 1 and 30 mg of MT, optionally 4-16 mg, to the subject per day, optionally split into 2 or more doses. For example the total daily dose is about 2, 3, 4, 5, 6, 7, 8, 9, 10, 11, 12, 13, 14, 15, 16, 17, 18, 19, 20, 21, 22, 23, 24, 25, 26, 27, 28, 29, or 30 mg.

Wherein the oral pharmaceutical composition comprises the buoyant sustained release capsule, it may be preferable that the composition does not need to be administered every day, or is at most administered once a day. This can provide benefits in patient compliance.

In one embodiment administration provides an amount of MT to the subject that corresponds to an average amount per day of from around any of 0.05, 0.1, 0.2, 0.3, 0.4, 0.5, 1, 1.5, and 2 mg to around any of 2.5, 3, 5, 6, 7, 8, 9, 10, 11, 12, 13, 14, 15, 16, 17, 18, 19, 20, 25 and 30 mg.

The subject of the present invention may be an adult human, and the dosages described herein are premised on that basis (typical weight 50 to 70 kg). If desired, corresponding dosages may be utilised for subjects outside of this range by using a subject weight factor whereby the subject weight is divided by 60 kg to provide the multiplicative factor for that individual subject.

In one embodiment a regimen employs a dosage between 0.05 mg and 40 mg MT per day, preferably between 0.1 mg and 20 mg MT per day, for a treatment period of at least 1 month.

For treatment of the neurodegenerative disorder of protein aggregation described herein, a treatment regimen based on the gastroretained MT compounds will preferably extend over a sustained period of time.

For example, the duration of treatment may be:

At least 1, 2, 3, 4, 5, 6, 7, 8, 9, 10, 11, 12 months, or longer.

At least 2, 3, 4, 5 years, or longer.

Between 6 and 12 months.

Between 1 and 5 years.

Where the disorder is AD, duration may be such as to achieve any one or more of:

A 3, 4 or 5-point improvement on the 11-item Alzheimer's Disease Assessment Scale-cognitive subscale (ADAS-cog) over a 52-week period;
4, 5 or 6 point improvement on the 23-item Alzheimer's Disease Cooperative Study Activities of Daily Living (ADCS-ADL) over a 52-week period;
A reduction in the increase of Lateral Ventricular Volume (LVV), as measured by the Ventricular Boundary Shift Integral (VBSI) of 1 or 2 cm³ over a 52-week period.

A decrease in annualized rate of whole brain atrophy on brain MRI using BSI.

For prophylaxis, the treatment may be ongoing.

As used herein the term ‘treating’, ‘treat’ or ‘treatment’ refers to stopping or reducing further sickness, wasting, infection or death in a human i.e. treating includes a reduction in the rate of progress, a halt in the rate of progress, regression of the condition, amelioration of the condition, and cure of the condition.

The term “therapeutically-effective amount,” as used herein, pertains to that amount of a composition or dosage from comprising the MT/LMT compound, which is effective for producing some desired therapeutic effect, commensurate with a reasonable benefit/risk ratio, when administered in accordance with a desired treatment regimen.

MT compound in respect of the diseases of the invention may be much lower than was hitherto understood in the art.

The invention also embraces treatment as a prophylactic measure is also included.

The term “prophylactically effective amount,” as used herein, pertains to that amount of a compound of the invention, or a material, composition or dosage from comprising said compound, which is effective for producing some desired prophylactic effect, commensurate with a reasonable benefit/risk ratio, when administered in accordance with a desired treatment regimen.

“Prophylaxis” in the context of the present specification should not be understood to circumscribe complete success i.e. complete protection or complete prevention. Rather prophylaxis in the present context refers to a measure which is administered in advance of detection of a symptomatic condition with the aim of preserving health by helping to delay, mitigate or avoid that particular condition.

The term “treatment” includes “combination” treatments and therapies, in which two or more treatments or therapies for the same neurodegenerative disorder of protein aggregation, are combined, for example, sequentially or simultaneously. These may be symptomatic or disease modifying treatments.

The particular combination would be at the discretion of the physician.

In combination treatments, the agents (i.e., an MT compound as described herein, plus one or more other agents) may be administered simultaneously or sequentially, and may be administered in individually varying dose schedules and via different routes. For example, when administered sequentially, the agents can be administered at closely spaced intervals (e.g., over a period of 5-10 minutes) or at longer intervals (e.g., 1, 2, 3, 4 or more hours apart, or even longer periods apart where required), the precise dosage regimen being commensurate with the properties of the therapeutic agent(s).

An example of a combination treatment of the invention would be an agent which is MT-containing compound at the specified dosage in combination with an agent which is an inhibitor of amyloid precursor protein to beta-amyloid (e.g., an inhibitor of amyloid precursor protein processing that leads to enhanced generation of beta-amyloid).

In other embodiments the treatment is a “monotherapy”, which is to say that the MT-containing compound is not used in combination (within the meaning discussed above) with another active agent for treating the same neurodegenerative disorder of protein aggregation in the subject.

In the present invention, when treating AD at least, it is preferred that the treatment does not include administration of either or both of: an acetylcholinesterase inhibitor or an N-methyl-D-aspartate receptor antagonist. The MT-compound based treatment of AD may optionally be a monotherapy.

The gastroretained compositions described herein (MT containing compound plus optionally other ingredients, or gastroretained MT composition more generally for treatment in AD) may be provided in a labelled packet along with instructions for their use.

In one embodiment, the pack is a bottle, such as are well known in the pharmaceutical art. A typical bottle may be made from pharmacopoeial grade HDPE (High-Density Polyethylene) with a childproof, HDPE pushlock closure and contain silica gel desiccant, which is present in sachets or canisters. The bottle itself may comprise a label and be packaged in a cardboard container with instructions for us and optionally a further copy of the label.

In one embodiment, the pack or packet is a blister pack (preferably one having aluminium cavity and aluminium foil) which is thus substantially moisture-impervious. In this case the pack may be packaged in a cardboard container with instructions for us and label on the container.

Said label or instructions may provide information regarding the neurodegenerative disorders of protein aggregation (e.g. cognitive or CNS disorder) for which the medication is intended.

Where the medication is indicated for AD, said label or instructions may provide information instructing the user that the compositions should not be used in conjunction with any of: an acetylcholinesterase inhibitor or an N-methyl-D-aspartate receptor antagonist.

Said label or instructions may provide information regarding the maximum permitted daily dosage of the compositions as described herein.

Said label or instructions may provide information regarding the suggested dosage regimen for the treatment, as described herein. For example, a suggested dosage frequency of every other day, twice weekly, once weekly, etc. The disclosure of dosage frequencies above thus applies mutatis mutandis to this aspect.

Said label or instructions may provide information regarding the suggested duration of treatment, as described herein. The disclosure of treatment duration above thus applies mutatis mutandis to this aspect.

A number of patents and publications are cited herein in order to more fully describe and disclose the invention and the state of the art to which the invention pertains. Each of these references is incorporated herein by reference in its entirety into the present disclosure, to the same extent as if each individual reference was specifically and individually indicated to be incorporated by reference.

Unless defined otherwise, all other technical and scientific terms used herein have the same meaning as is commonly understood by a skilled person to which the subject matter herein belongs.

Throughout this document, unless otherwise indicated to the contrary, the terms “comprising”, “consisting of”, “having” and the like, are to be construed as non-exhaustive, or in other words, as meaning “including, but not limited to”.

It must be noted that, as used in the specification and the appended claims, the singular forms “a,” “an,” and “the” include plural referents unless the context clearly dictates otherwise. Thus, for example, reference to “a pharmaceutical carrier” includes mixtures of two or more such carriers, and the like.

Furthermore, throughout the document, unless the context requires otherwise, the word “include” or variations such as “includes” or “including” will be understood to imply the inclusion of a stated integer or group of integers but not the exclusion of any other integer or group of integers

Ranges are often expressed herein as from “about” one particular value, and/or to “about” another particular value. When such a range is expressed, another embodiment includes from the one particular value and/or to the other particular value. Similarly, when values are expressed as approximations, by the use of the antecedent “about,” it will be understood that the particular value forms another embodiment.

Any sub-titles herein are included for convenience only, and are not to be construed as limiting the disclosure in any way.

The invention will now be further described with reference to the following non-limiting Figures and Examples. Other embodiments of the invention will occur to those skilled in the art in the light of these.

The disclosure of all references cited herein, inasmuch as it may be used by those skilled in the art to carry out the invention, is hereby specifically incorporated herein by cross-reference.

### FIGURES

FIG. 1: oral formulation (a) in solution and (b) in a solid matrix. The oral formulation as depicted in [FIG. 1b] is in a solid form including that of a tablet 2 with a gastroretentive platform that releases the effective amount of diaminophenothiazine compound in a controlled sustained release manner targeting the upper gastrointestinal tract especially for human use. In various embodiments as depicted in [FIG. 1b]diaminophenothiazine compound such as MTC is encapsulated in microparticles 4 and then encompassed in a rapid release matrix 6. The rapid release matrix 6 may be formed of elements that dissolve in low pH allowing the diaminophenothiazine loaded microparticle to be disbursed throughout and held in place within the matrix but not released until it reaches the stomach. The lower pH in the stomach then allows for a rapid release of the diaminophenothiazine loaded microparticles 4 within the stomach.

FIG. 2: A bird (A) before and (B) after treatment for avian megalobacteriosis (C) having only 2% of the original megabacteria pathogen load in stool smear microscopy after treatment.

FIG. 3: Comparison between “minipill” of the invention and standard capsule.

FIG. 4: A floating MTC minipill of the invention, approximately 60 seconds after being applied to the surface of potable water.

The pill floated for around 150 seconds by which time is was largely dissolved. By contrast a non-floating minipill (of similar dimensions and density, but lacking the coating) sank within approximately 10 seconds (not shown).

FIG. 5: Schematic illustration of a coated capsule employing Soctec® technology.

FIG. 6: (A) Uncoated vs. final aspect of the coated capsule, which is dark blue, with a visually homogeneous film coating; (B) uncoated vs. coated capsules (layer 1 and layer 1+layer 2).

FIG. 7: Flowchart showing capsule layer 1 application process.

FIG. 8: Flowchart showing capsule layer 2 application process.

FIG. 9: Dissolution profile of 6 coated MTC capsules in one media (buffer pH 1.2, USP), taking into account capsule mass.

FIG. 10: Dissolution profile of 6 coated MTC capsules in one media (buffer pH 1.2, USP), not taking into account capsule mass.

FIG. 11: Density measurement of tablets. Cylinder containing ethylene glycol before (a) and after (b) the tablet addition.

FIG. 12: Flowchart summarising the manufacturing process

FIG. 13: Flowchart summarising the manufacturing process

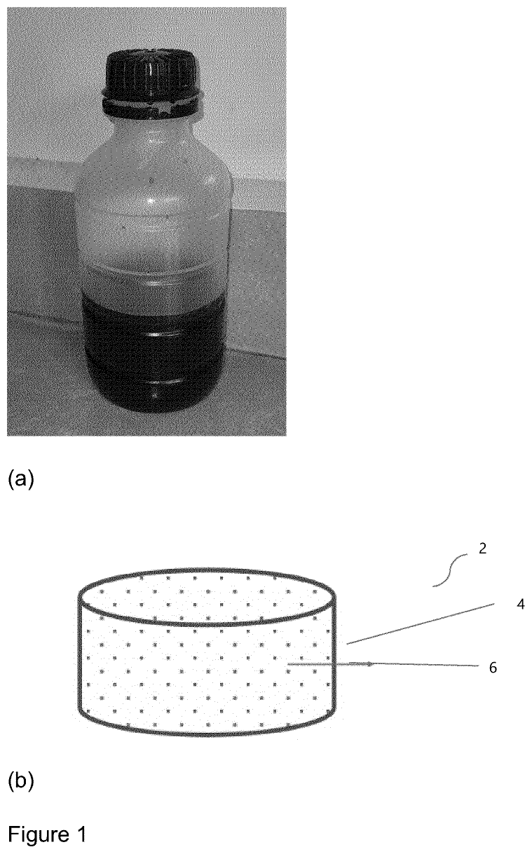

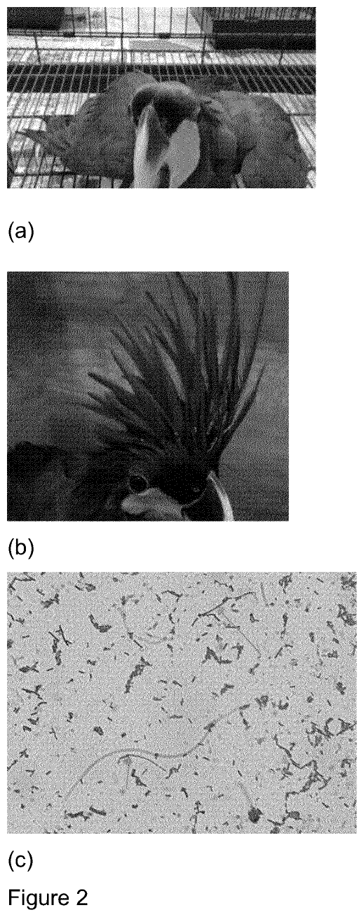

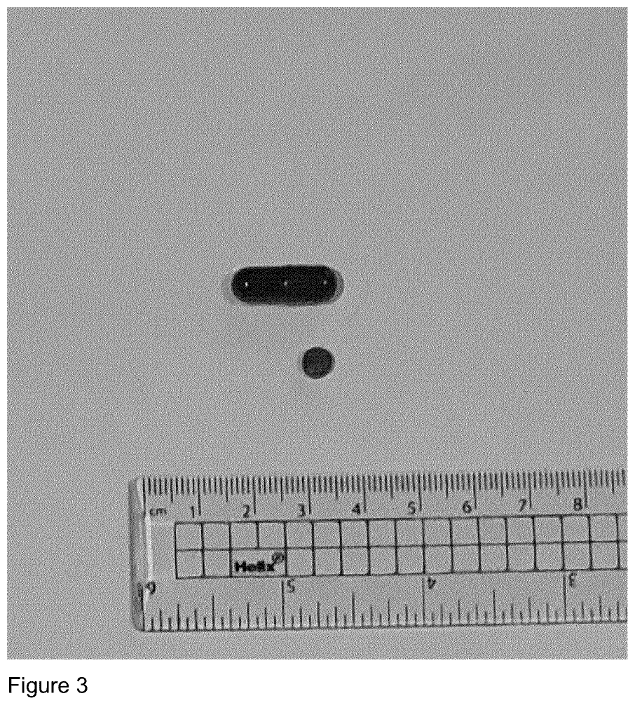

### EXAMPLES

### Example 1: Treatment of Avian Meglobacteriosis with MTC Dosage Forms

A black palm cockatoo was diagnosed by a vet with megabacteria infection. The bird was pale, crop was distended, stools were liquid and contained undigested food. This resulted in weight loss of 800 mg from pre-morbid conditions. Given the poor condition the bird was prescribed with palliative supplement management (Papaya seed, juice, formula, and supplements).

The bird was treated for 54 days with a daily dose of 0.05 ml of oral formulation (0.003% MTC and 0.008% hyaluronic acid in sterile water).

Without being limited to any theory the inventor postulates that the hyaluronic acid and/or chitosan forms an attachment to mucosal lining of the stomach or duodenum wall holding the active ingredient of DAPTZ around this area for an extended time.

The black palm cockatoo's condition over the course of treatment is summarized in the following Table:

| | TREATMENT | WEIGHT | |
|---|---|---|---|
| DATE | DAY | (grams) | CONDITION |
| 4 Mar. 2022 | Pre-treatment | 620 | pale, crop was distended, stools were liquid |
| | (supplement | | and contained undigested food |
| | management) | | |
| 19 Mar. 2022 | Pre-treatment | 615 | Pallor increase, more active |
| | (supplement | | |
| | management) | | |
| 23 Mar. 2022 | Pre-treatment | 635 | Melaena (dark black, tarry feces) |
| | (supplement | | |
| | management) | | |
| 24 Mar. 2022 | 1 | 635 | Melaena |
| 26 Mar. 2022 | 3 | 700 | No more melaena, diarrhea, appetite |
| | | | improved |
| 27 Mar. 2022 | 4 | 735 | Good appetite, well-formed stools |
| 28 Mar. 2022 | 5 | 760 | Good appetite, well-formed stools |
| 30 Mar. 2022 | 7 | 765 | Change to increase seeds in diet |
| 6 Apr. 2022 | 13 | 755 | Very active and well |
| 18 Apr. 2022 | 25 | 735 | Very well and increased activity, |
| | | | proventriculus no longer dilated |
| 28 Apr. 2022 | 35 | 750 | Healthy appetite, weight gain, energetic |
| 4 May 2022 | 41 | 760 | Healthy appetite, weight gain, energetic |
| 12 May 2022 | 49 | 775 | Healthy appetite, weight gain, energetic |
| 17 May 2022 | 54 (last day) | 775 | Healthy appetite, weight gain, energetic |
| 24 May 2022 | Post | 785 | Healthy appetite, weight gain, energetic |
| | treatment | | |
| 25 May 2022 | Post | 790 | Detection of 2% megabacteria in stool smear |
| | treatment | | microscopy |
| 7 Jun. 2022 | Post | 800 | Well and thriving |
| | treatment | | |
| 20 Jun. 2022 | Post | 810 | Remains well and thriving |
| | treatment | | |

### Example 2: Treatment of Avian Bornavirus Infection with MTC Dosage Form

A Moluccan cockatoo presented with regurgitation, poor appetite, indigestion and lack of energy. A PCR test on a cloacal smear tested positive for avian bornavirus (ABV).

The bird was treated for 43 days with a daily dose of 0.15 ml of oral formulation (0.003% MTC and 0.008% hyaluronic acid in sterile water) each morning.

The Moluccan cockatoo's condition over the course of treatment is summarised in the following table.

| | TREATMENT | |
|---|---|---|
| DATE | DAY | CONDITION |
| 30 Mar. 2022 | Pre-treatment | regurgitation, poor appetite, |
| | | indigestion and lack of energy |
| 9 Jun. 2022 | 1 | poor appetite, lack of energy, |
| | | indigestion |
| 12 Jun. 2022 | 3 | Improved with less indigestion, |
| | | more energetic |
| 21 Jul. 2022 | 43 (last day) | Healthy appetite, weight gain, |
| | | energetic |
| 18 Aug. 2022 | Post treatment | Negative for PCR detection of |
| | | ABV in cloacal smear |

### Example 3: Treatment of Parrot Beak and Feather Disease

A Moluccan cockatoo presented with regurgitation, poor appetite, indigestion and lack of energy. The bird was diagnosed with a positive PCR test on a blood sample for psittacine circovirus (PCV) that causes parrot beak and feather disease.

The bird was treated for 43 days with a daily dose of 0.15 ml of oral formulation (0.003% MTC and 0.008% hyaluronic acid in sterile water) each morning.

The Moluccan cockatoo's condition over the course of treatment is summarised in the following table.

| | TREATMENT | |
|---|---|---|
| DATE | DAY | CONDITION |
| 30 Mar. 2022 | Pre-treatment | regurgitation, poor appetite, |
| | | indigestion and lack of energy |
| 9 Jun. 2022 | 1 | poor appetite, lack of energy, |
| | | indigestion |
| 12 Jun. 2022 | 3 | Improved with less indigestion, |
| | | more energetic |
| 21 Jul. 2022 | 43 (last day) | Healthy appetite, weight gain, |
| | | energetic |
| 11 Aug. 2022 | Post | Negative for PCR detection of |
| | treatment | PCV in a blood sample |

### Example 4: Prevention of Avian Megalobacteriosis

The black palm cockatoo diagnosed by a vet with megabacteria infection in Example 1 was co-habiting an area with 1 other cockatoo within the same cage. The infected bird had a high amount of megabacteria in its stool meaning the co-habiting bird had a high chance of coming into contact with the infection and being infected by Macrorhabdus ornithogaster. It was initially suggested that the co-habiting bird should be culled to prevent any further spread of the megabacteria infection. As an alternative, the bird was administered a weekly dose of 0.05 ml of oral formulation (0.003% MTC and 0.008% hyaluronic acid in sterile water) until the estimated incubation period of the pathogen was over and the infective source has been resolved.

As a result of the preventative weekly prophylactic the co-habiting bird did not show any signs of infection and remained healthy:

| | TREATMENT | |
|---|---|---|
| DATE | DAY | CONDITION |
| 23 Mar. 2022 | Pre-treatment | Co-habiting partner of infected |
| | | Black palm cockatoo. Remains |
| | | Well and asymptomatic |
| 24 Mar. 2022 | 1 | Remains Well and asymptomatic |
| 26 Mar. 2022 | 3 | Remains Well and asymptomatic |
| 26 May 2022 | Last day of | Remains Well and asymptomatic |
| | treatment | |

### Example 5—Preparation of Floating MTC Minipills

Synthesis of MTC is well known in the art. Examples syntheses of highly pure MTC are provided in WO2006/032879 and WO2008/007074.

The 4-mg MTC tablets were formulated as blue, film-coated, immediate release tablets debossed with the Greek tau (“τ”) symbol on one side.

The 4-mg MTC placebos were compressed using 5 mm round tooling, resulting in corresponding dimensions.

In addition to MTC the MTC tablets also contained the same inactive ingredients as the 4-mg IMP LMTM tablets: mannitol (Pearlitol® 200 SD), Ph.Eur. and USP; microcrystalline cellulose, Ph.Eur. and NF; crospovidone, Ph.Eur. and NF; magnesium stearate (vegetable source), Ph.Eur. and NF; and Opadry® II 85G205011 Blue.

After manufacture the 4-mg MTC placebo tablets were packaged in aluminium foil cold-form blisters and stored at <25° C.

TABLE 1
Composition of 4-mg MTC Tablets

| | | Placebo for |
|---|---|---|
| | | LMTM Tablet |
| Component | | Composition |
| Tablet core | Function | (mg/tablet) |
| MTC, Ph. Eur. | API | 4.00¹ (5.76) |
| Mannitol (Pearlitol ® 200 SD), Ph. Eur. | Diluent | 36.40 (34.64)¹ |
| Microcrystalline cellulose, Ph. Eur. | Diluent | 12.00 |
| Crospovidone, Ph. Eur. | Disintegrant | 2.00 |
| Magnesium stearate (vegetable source), | Lubricant | 0.60 |
| Ph. Eur. | | |
| Total tablet core weight | | 55.00 |
| Opadry II Blue 85G205011** | Film coat | 2.75 |
| Total Tablet Weight | | 57.75 |

¹MT⁺ content - the quantity of mannitol was adjusted to take into account total weight of MTC
**The 4-mg MTC tablets were coated with an Opadry ® II Blue 85G205011 (Colorcon)/water mixture.

In order to ensure the fluid was uniformly applied throughout the film-coating process, the tablets were manufactured using an overage of coating fluid. This enabled essential mixing of the coating fluid during application of the film coat and, once the requisite amount of coating fluid had been sprayed, the remaining coating mixture was discarded.

The 4-mg MTC tablets were manufactured using a dry mixing process. The excipients were blended followed by a final lubrication step with magnesium stearate.

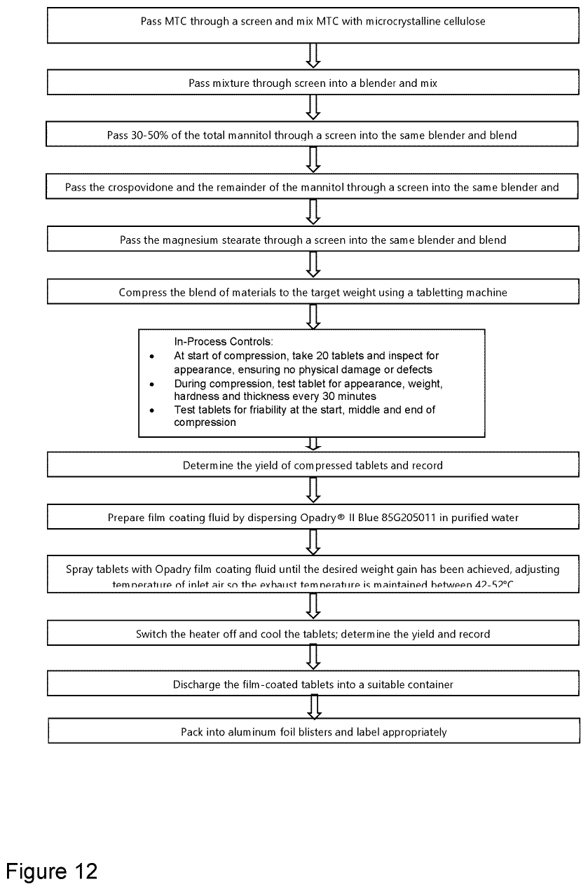

A flowchart summarising the manufacturing process is shown in FIG. 12.

### Friability Testing

Friability was tested using commercially available instruments (Erweka, or Copley) calibrated before use. Samples were measured as 10 tablets or approximately 6.5 g of tablets over a 4 minute duration. Friability was expressed as:

$$Friability \% = ( initial weight ( g ) - final weight ( g ) ) \times 100 \% / initial weight ( g )$$

### Hardness Testing

Hardness (in kp, kilopond) was tested using a commercially available instrument (Erweka TBH class) according to the manufacturers instructions, and calibrated before use.

### Disintegration

To carry out a disintegration test for tablets a tablet was placed in a basket in a disintegration tube. The basket is lowered into the tube of water. The baskets are raised each minute and the tablets observed. The tablet disintegration time is taken when either no residue is left in the mesh, or when the residue is a soft mass with no palpably firm core.

### Tablet Density

100 tablets were counted out and weighed. A 100 mL measuring cylinder was filled with 50 mL ethylene glycol. The volume of ethylene glycol was recorded before and after the tablets were added to the measuring cylinder. The cylinder was tapped and shaken after addition of the tablets to ensure there were no air bubbles remaining. Density was calculated by dividing the weight of tablets (g) by the difference in volume.

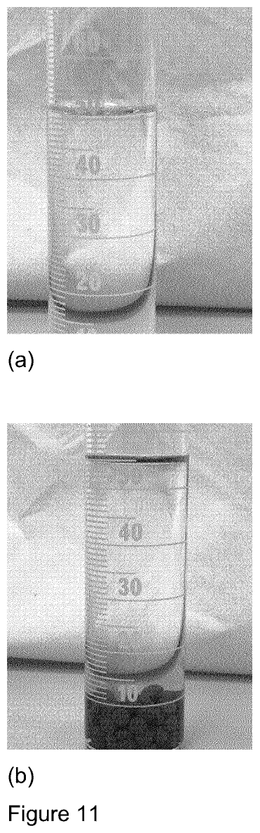

FIG. 11 shows the cylinder before (a) and after (b) the tablet addition.

| | | | | Volume of | |
|---|---|---|---|---|---|
| | | Total weight | Volume of | ethylene | |
| Tablet | Batch | of 100 | ethylene | glycol and | Density |
| strength | number | tablets (g) | glycol (mL) | tablets (mL) | (g/mL) |
| 4 mg | B17433 | 5.8084 | 50 | 54.5 | 1.291 |
| 8 mg | B24777 | 8.3804 | 50 | 56.5 | 1.290 |

The 4-mg MTC tablets have the following specification:

| | | Results | |
|---|---|---|---|
| Test | Specification | Batch B22632 | Batch B24493 |
| Appearance | A round blue | A round blue | A round blue |
| | coloured film | coloured film | coloured film |
| | coated tablet with | coated tablet with | coated tablet with |
| | ‘τ’ debossed on | ‘τ’ debossed on | ‘τ’ debossed on |
| | one side | one side | one side |
| Disintegration | NMT 20 minutes | All tablets had | All tablets had |
| | | disintegrated within | disintegrated within |
| | | 3 minutes | 3 minutes |
| Water content | Report results | 2.44% w/w | 2.76% w/w |
| Average tablet | Report results | 58.05 mg (1.7% | 58.00 mg (0.6% |
| weight | | RSD) | RSD) |
| Identification by | Retention time of main | ID confirmed | ID confirmed |
| retention time | peak in sample is | | |
| | within ±5% of the | | |
| | retention time in the | | |
| | standard solution | | |
| Assay (MT free | 85.0%-115.0% of label | 98.0% LC | 100.4% LC |
| base) | claim | | |
| Related | | | |
| substances | | | |
| Azure B | ≤2.0% w/w | 0.92% w/w | 0.45% w/w |
| Each other | ≤0.5% w/w | Azure A: | Azure A: |
| individual | | 0.05% w/w | 0.06% w/w |
| impurity | | | |
| Total impurities | ≤3.0% w/w | 0.97% w/w | 0.51% w/w |
| Uniformity of | | | |
| Dosage Units | | | |
| Weight Variation | Complies with USP | AV 4.4 | AV 1.5 |
| (coated tablets) | <905> Ph. Eur. 2.9.40 | | |
| Content | Complies with USP | AV 5.6 | AV 3.5 |
| Uniformity | <905> Ph. Eur. 2.9.40 | 99.6% LC (2.4% | 100.7% LC (1.4% |
| (tablet cores) | with L1 = 25.0 | RSD; 2.3 SD) | RSD; 1.4 SD) |
| Total aerobic | NMT 1000 cfu/g | <100 cfu/g | <100 cfu/g |
| microbial count | | | |
| Total yeast and | NMT 100 cfu/g | <50 cfu/g | <50 cfu/g |
| mould count | | | |
| Specified | Absence of | Absent | Absent |
| microorganisms | E. coli | | |

### Example 6—Preparation of Floating HMTM Minipills

Synthesis of compound LMTM can be performed according to the methods described in WO2007/110627, or a method analogous to those.

The LMTM tablets used in clinical trials were provided in one strength: 4 mg (potency expressed as the LMT free base equivalent), formulated as 5 mm round, blue film-coated tablets debossed with the Greek tau symbol (τ) on one side. Tablets are packaged in aluminium blister packages or HDPE bottles and protected from moisture and light.

In addition to the API, the LMTM pill contains the following inactive ingredients: mannitol (Pearlitol® 200 SD), Ph.Eur.; microcrystalline cellulose, Ph.Eur.; crospovidone, Ph.Eur.; magnesium stearate (vegetable source), Ph.Eur.; and Opadry® II 85G205011 Blue (supplied by Colorcon Limited).

TABLE 2
Composition of 4-mg LMTM Tablets

| | | Composition |
|---|---|---|
| Components | | (mg/tablet) |
| Tablet Core | Function | 4 mg |
| LMTM | API | 4.001 (6.69)² |
| Mannitol (Pearlitol ® 200 SD), Ph. Eur. | Diluent | 36.401 (33.71)³ |
| Microcrystalline cellulose, Ph.Eur | Diluent | 12.00 |
| Crospovidone, Ph. Eur. | Disintegrant | 2.00 |
| Magnesium stearate (vegetable source), | Lubricant | 0.60 |
| Ph. Eur. | | |
| Total Tablet Core Weight | | 55.00 |
| Film Coat: | Film coat | |
| Opadry ® Il Blue 85G205011 | | |
| Total Film Coat Weight | | 2.756 |
| Total Tablet Weight (mg) | | 57.75 |

¹ Theoretical: API expressed as the free base equivalent. The quantity of mannitol was adjusted accordingly.
²Present as the pure salt (conversion from the drug base)
³The quantity of mannitol used is adjusted according to the amount of LMTM present in batch. This amount varies from batch to batch depending on the purity of the salt.

All excipients in the tablet core of the LMTM formulation comply with Ph.Eur. and NF/USP monographs. Specifically, these excipients are mannitol, microcrystalline cellulose, crospovidone, and magnesium stearate (vegetable source).

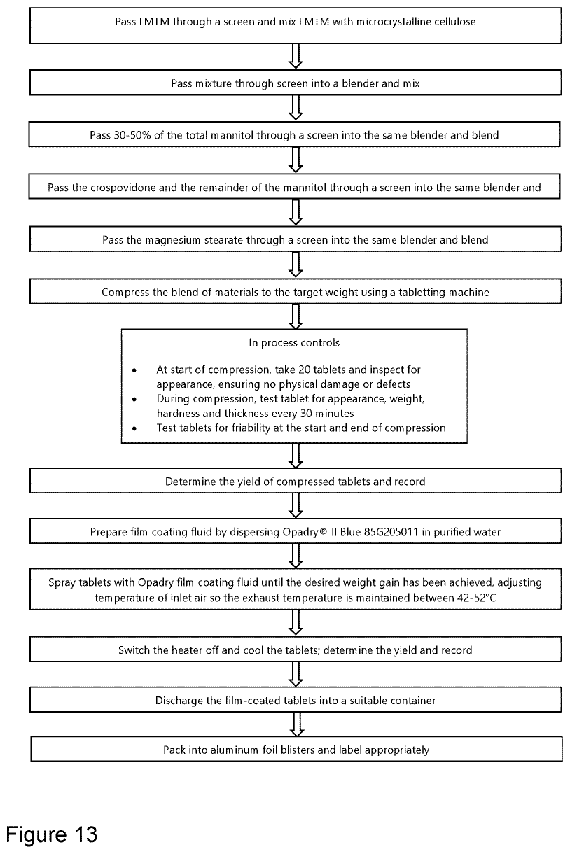

A flowchart summarising the manufacturing process is shown in FIG. 13.

### Example 7—Properties and Clinical Efficacy of MTC Minipills

Prior-filed PCT/EP2023/064369 (Example 3) describes the structure of a phase 3 clinical trial in mild to moderate AD in which different arms of participants were treated with MTC and LMTM respectively. This was a Phase 3, randomized, double-blind, outpatient trial to evaluate the safety, efficacy, and tolerability of LMTM in participants with severity ranging from mild cognitive impairment (MCI) to moderate AD. The MTC minipill was intended as a urinary discolourant placebo (in combination with a true placebo i.e. a blank minipill lacking any MTC or LMTM). Unexpectedly a group from the arm taking the 4 mg MTC (approximately twice weekly) showed clinical benefit despite receiving such a small dose of drug. Of patients receiving MTC 4 mg twice weekly, the majority (85%) were unexpectedly found to have blood levels of active drug above the threshold needed to produce a clinical effect.

It appears that 4 mg MTC twice weekly leads to accumulation of MT in these patients indicating a high level of absorption (and conversion to LMT).

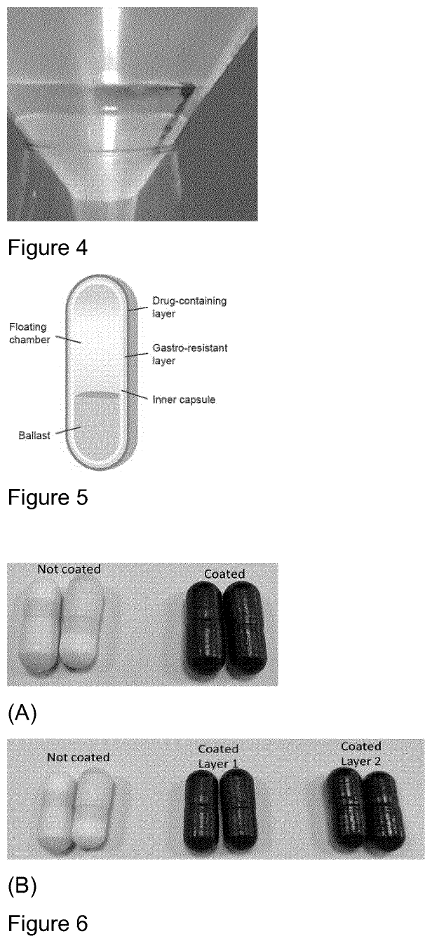

Further investigation into the physical properties of the MTC tablets revealed that they unexpectedly floated in in vitro tests, while rapidly dissolving (see FIG. 4 and legend).

Notably the gastric contents have a density close to water (c. 1.004 g/cm3—see Aute, Swati M., et al. “Novel approach in gastro retentive drug delivery system: Floating Microspheres.” International Journal of Pharmacy and Biological Sciences Archive 2.5 (2014): 09-22.). Furthermore it is been reported that the floating characteristics of dosage form excipients is typically improved in simulated gastric fluid compared to deionized water (Gerogiannis, V. S., et al. “Floating and swelling characteristics of various excipients used in controlled release technology.” Drug development and industrial pharmacy 19.9 (1993): 1061-1081). Therefore it can be concluded that these tablets float in vivo. Since the calculated density of the tablets was greater than 1, a key feature of the floating property is believed to be the hydrophilic macromolecule (e.g. PVA) present in the coating, which coating is equal to greater than 5% by weight of the tablet.

Without wishing to be bound by theory, it is believed that the minipills float at the surface of the gastric fluid, with the low pH permitting rapid release of the MTC compound, which leads to the released DAPTZ compound being maximally exposed to maximal surface area of the stomach mucosal lining in the acidic stomach environment, thereby providing optimal gastro-targeted absorption, and enhancing accumulation to therapeutic levels.

### Example 8—Floating Gastro-Retentive MTC Capsules

### 8.1 Introduction

This Example describes production of a gastroretentive capsule using SOCTEC® (Self Orienting Capsule Technology; see WO2012004231). Soctec® technology is a gastrointestinal drug delivery system that combines a hydrodynamically balanced flotation system with a shape that prevents premature stomach expulsion. In the present case the system was specifically adapted to release between 5 and 20 mg of MT from MTC (referred to herein as “MB” or “Methylene Blue”) in the stomach during the gastric residence time such as to maximise reduction of MT+ and absorption of LMT in the upper gastrointestinal tract. In this Example the aim was to unload approximately 16 mg of Methylene Blue (MB) in total during the gastric residence time.

### 8.2 Manufacture of Soctec™ Carrier

After ingestion, the Soctec® capsule immediately floats upright and does not pass through the bottom of the stomach. The buoyancy chamber is counterbalanced by a ballast (see FIG. 5).

The ballast selected was a round, flat tablet of barium sulfate, which was granulated, then dried and milled. The resulting milled granules was then blended with excipients.

The ballast tablet was then filled into a size 3 capsule (typical diameter of about 5.8 mm and a locked length of about 15.9 mm). The filled capsule is then filled into a size 00 capsule (typical diameter of about 8.5 mm and a locked length of about 23.3 mm). This capsule is then coated with a gastro-resistant film, enabling it to remain in the stomach for at least 10 hours.

### 8.3 Composition

The first coating layer of the Soctec® prototype was composed of the colouring/API and polymers intended to provide for sustained release. Two polymers were selected for this specific API: Eudragit® RS 30D which is hydrophobic polymer and Eudragit® RL 30D which is hydrophilic polymer.

The second coating layer is composed only of the polymers. The second layer is intended to slow down MB colour loss and eventual release.

### 8.4 Batch Quantitative and Qualitative Formula

The composition of the two layers is described in the following table:

TABLE 3
proportions of coating suspensions:

| | Ingredients | | Mass proportion |
|---|---|---|---|
| | FIRST | Methylene Blue | 2.50% |
| | LAYER | Eudragit ® RS 30D | 24.00% |
| | | Eudragit ® RL 30D | 6.00% |
| | | TEC (TriEthyl Citrate) | 2.00% |
| | | Talc | 5.00% |
| | | Water | 60.50% |
| TOTAL | | | 100.00% |
| | SECOND | Eudragit ® RS 30D | 8.00% |
| | LAYER | Eudragit ® RL 30D | 32.00% |
| | | TEC | 2.35% |
| | | Talc | 5.88% |
| | | Water | 51.77% |

| | |
|---|---|
| TOTAL | 100.00% |

### 8.5 Manufacturing Process

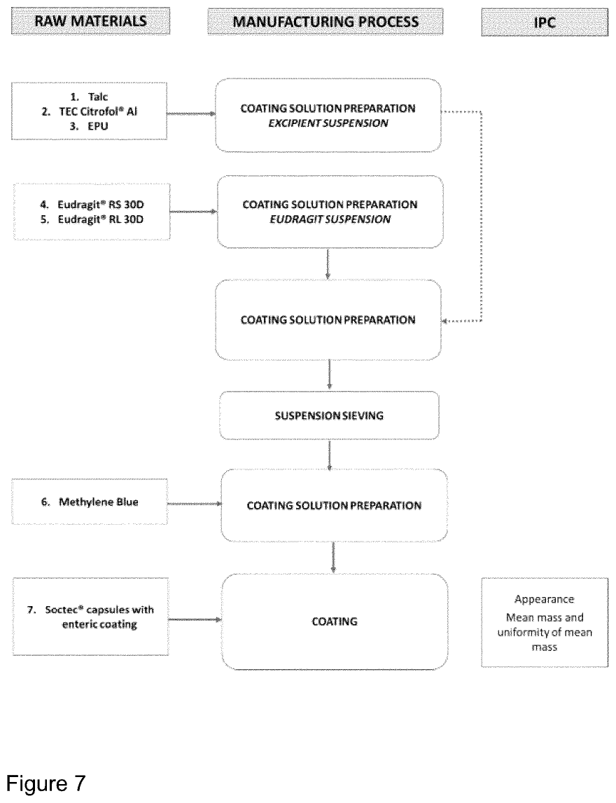

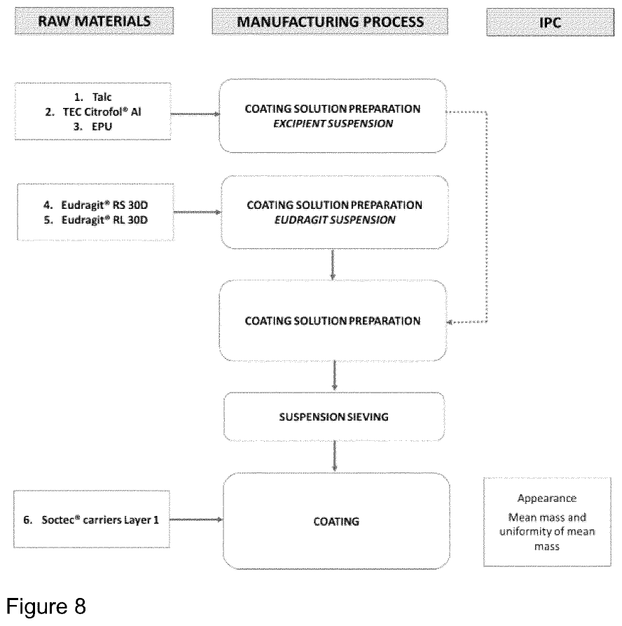

The manufacturing steps of the capsule are summarized in the Flowcharts in FIGS. 7 and 8.

### 8.5.1 Composition of the Soctec® Prototype

The first layer was composed of Methylene Blue, sustained release polymers, TEC and talc. The total mass gain of the capsule for this coating layer was calculated to obtain 16 mg of colouring on the capsule. The second layer applied was composed of sustained release polymers, TEC and talc. Some capsules of prototype were taken before applying the second layer.

| | | Amount in | Amount on |
|---|---|---|---|
| | % | solution | one capsule |
| Ingredients | (w/w) | (g) | (mg) |
| Layer 1 | | | |
| Methylene Blue (API) | 2.50% | 6.26 | 14.06 |
| Eudragit ® RS 30D | 24.00% | 59.99 | 40.36 (dry |
| in solution | | | Eudragit) |
| Eudragit ® RL 30D | 6.00% | 15.01 | 10.11 (dry |
| in solution | | | Eudragit) |
| TEC | 2.50% | 4.99 | 11.21 |
| Talc | 5.00% | 12.50 | 28.07 |
| Water | 60.50% | 151.24 | |
| Total | 100.00% | 249.99 | 103.81 |
| Mass gain (%) | 13.86% | | |
| Layer 2 | | | |
| Eudragit ® RS 30D | 8.01% | 8.02 | 1.98 (dry |
| in solution | | | Eudragit) |
| Eudragit ® RL 30D | 31.98% | 32.01 | 7.92 (dry |
| in solution | | | Eudragit) |
| TEC | 2.36% | 2.36 | 1.95 |
| Talc | 5.89% | 5.89 | 4.86 |
| Water | 51.76% | 51.80 | |
| Total | 100.00% | 100.08 | 16.70 |

| | |
|---|---|
| Mass gain (%) | 2.23% |

### 8.5.2 First Layer (Layer 1)

### 8.5.2.1 Coating Suspension Preparation

### 8.5.2.1.1 Excipient Suspension

Water and TEC were weighed in the same beaker. The agitation was assured by a paddle shaker and adjusted to form a vortex. While maintaining the vortex, talc was then added to the suspension. Agitation was maintained at least 10 minutes. A milky suspension was obtained. The table hereunder summarized the parameters of the suspension preparation:

Parameters for Excipient

| suspension preparation | 2314BLU01-C1 | |
|---|---|---|
| Speed during introduction | 550-580 | rpm |
| Introduction time | 5 | min |
| Speed during homogenizing time | 580 | rpm |
| Mixing time | 16 | min |

| | |
|---|---|
| Appearance | Homogeneous and milky suspension |

### 8.5.2.1.2 Coating Suspension (Eudragit®+Excipients)

A premix of Eudragit® RS 30D and Eudragit® RL 30D was stirred at 150 rpm for 2 minutes. The premix was added to the suspension of excipients with stirring. The resulting suspension was sieved on a 0.420 mm grid. After stirring at 280 rpm, suspension was homogenous and milky. The parameters of the coating suspension preparation are summarized in the following table.

| Parameters for Coating | |
|---|---|
| suspension preparation | 2314BLU01-C1 |

| | | |
|---|---|---|
| Mixing speed of the Eudragit ® premix | 250-280 | rpm |
| Mixing time of the Eudragit ® premix | 2 | min |
| Speed of addition of the premix to the | 280 | rpm |
| suspension | | |

| | |
|---|---|
| Addition Time | 3 min 40 s |
| Appearance | Homogeneous and |
| | milky suspension |

### 8.5.2.1.3 Methylene Blue Addition

Methylene Blue was then added to the sieved suspension. Agitation was maintained until total MB solubilisation. The table hereunder summarized the parameters of the suspension preparation.

| Parameters for MB addition | 2314BLU01-C1 | |
|---|---|---|
| Speed during addition | 450-480 | rpm |
| Addition time | 3 min 16 s | |
| Speed during homogenizing time | 480 | rpm |
| Mixing time | 19 | min |

| | |
|---|---|
| Appearance | Homogeneous and dark blue suspension |

A homogeneous, dark blue suspension with little foam on the surface was obtained.

### 8.5.2.2 Coating Parameters Setting

The table hereunder summarizes the coating parameters.

| Coating parameters | 2314BLU01-C1 | |
|---|---|---|
| Dry matter concentration | 18.50% | |
| Weight gain | 13.86% | |
| Product temperature | 30.6-41.7° | C. |
| Inlet air temperature | 49-52° | C. |
| Outlet air temperature | 33.2-38.9° | C. |
| Drum speed | 30 | rpm |
| Spray rate | ~3.6 | g/min |
| Sprayed solution quantity | 172.5 | g |

### 8.5.2.3 Coating of Capsules

The coating was performed in four steps:
1. Pre-heating of capsules to obtain a product temperature of 35-40° C. After the pre-heating, 30 capsules were sampled and weighed in order to determine the initial average mass of the capsules. These capsules were then reintroduced into the drum.
2. Spraying of the coating suspension until the calculated weight gain was achieved.
3. Drying the capsules for 30 minutes to form the sustained polymeric film.
4. Cooling the capsules until a product temperature of 30° C. was obtained.

The spraying was carried out in three parts. The process had to be interrupted twice because the spray nozzle became clogged. Each time, the spray nozzle was dismantled and cleaned. When the second clogging occurred, it was decided to filter the suspension through a 420 μm sieve to avoid any re-clogging of the nozzle.

### 8.5.2.4 Characterisation of Coated Capsules Manufactured

Non-coated and coated capsules are shown in FIG. 6 (a).

At the end of coating process, 30 capsules were sampled and weighted, in order to determine the final average weight as well as the uniformity of mean mass. Results are summarized in the table below:

| Determination of final weight | |
|---|---|
| average | 2314BLU01-C1 |
| Weight of 10 coated capsules - 1 | 8.5839 g |
| Weight of 10 coated capsules - 2 | 8.5274 g |
| Weight of 10 coated capsules - 3 | 8.5385 g |
| Mean final weight | 0.8550 g |

### 8.5.2.5 Process Mass Losses

Yield and reconciliation of coating process are presented below. Total losses from the manufacture of capsules are negligible.

| Batch | 2314BLU01-C1 | |
|---|---|---|
| Initial average tablet weight (PMI) | 750.9 | mg |
| Final average tablet weight (PMF) | 855.0 | mg |
| Weight gain (GM = (PMF − PM1)/PM1 × 100) | 13.86% | |
| Quantity of sprayed solution (QSP) | 172.5 | g |
| Dry matter solution concentration (MS) | 18.50% | |
| Dry matter sprayed (MSP = QSP * MS/100) | 31.91 | g |
| MB quantity sprayed on one capsule | 14.06 | mg |
| Mass of uncoated capsules (CR) | 200.05 | g |
| Masse capsules in destruction (CD) | N/A | |
| Mass of coated capsules (CP) | 211.88 | g |
| Sampling mass (PR) | 8.52 | g |

| | |
|---|---|
| Yield ((CP + PR)/(CR + MSP) × 100) | 95.0% |
| Reconciliation ((CP + CD + PR)/(CR + MSP) × 100) | 95.0% |

Final MB quantity sprayed on one capsule was determined with the calculated weight gain.

### 8.5.3 Second Layer (Layer 2)

A second layer coating was applied. A delay of 24h was respected between the first and the second coating manufacture.

### 8.5.3.1 Coating Suspension Preparation

### 8.5.3.1.1 Excipient Suspension

Water and TEC were weighed in the same beaker. The agitation was assured by a paddle shaker and adjusted to form a vortex. While maintaining the vortex, talc was then added to the suspension. Agitation was maintained at least 10 minutes. A homogenous and milky suspension was obtained. The table hereunder summarized the parameters of the suspension preparation.

Parameters for Excipient

| suspension preparation | 2314BLU01-C2 | |
|---|---|---|
| Speed during introduction | 316-747 | rpm |
| Introduction time | 50 | s |
| Speed during homogenizing time | 450 | rpm |
| Mixing time | 15 | min |

| | |
|---|---|
| Appearance | Homogeneous and milky suspension |

### 8.5.3.1.2 Coating Suspension (Eudragit®+Excipients)

The premix of Eudragit® RS 30D and Eudragit® RL 30D was added to the suspension of excipients with stirring. The resulting suspension was sieved on a 0.420 mm grid. After homogenisation at 200 rpm, suspension was homogeneous and milky. The parameters of the coating suspension preparation are summarized in the following table.

| Parameters for Coating | |
|---|---|
| suspension preparation | 2314BLU01-C2 |

| | | |
|---|---|---|
| Mixing speed of the Eudragit ® premix | 150 | rpm |
| Mixing time of the Eudragit ® premix | 1 | min |
| Speed of addition of the premix to the | 350 | rpm |
| suspension | | |
| Addition Time | 2 | min |

| | |
|---|---|
| Appearance | Homogeneous and |
| | milky suspension |

### 8.5.3.2 Coating Parameters Setting

The table hereunder summarizes the coating parameters retained.

| Coating's parameters | 2314BLU01-C2 | |
|---|---|---|
| Dry matter concentration | 20.23% | |
| Weight gain | 2.23% | |
| Product temperature | 32.3-35.3° | C. |
| Inlet air temperature | 44° | C. |
| Outlet air temperature | 32.6-36.6° | C. |
| Drum speed | 30 | rpm |
| Spray rate | ~1.8 | g/min |
| Sprayed solution quantity | 34 | g |

### 8.5.3.3 Coating of Capsules

As layer 1, the layer 2 was coated performed in four steps:
1. Pre-heating of capsules until a product temperature of 40° C.
2. Spraying of the coating suspension until the calculated weight gain was achieved.
3. Drying the capsules for 20 minutes to form the sustained polymeric film.
4. Cooling of the capsules until a product temperature of 30° C. was obtained.

The final aspect of the capsule is dark blue, with a visually homogeneous film coating (FIG. 6(b)).

### 8.5.3.4 Characterisation of Manufactured Coated Capsules

At the end of each coating process, 30 capsules were sampled and weighted, in order to determine the final average weight as well as the uniformity of mean mass. Results are summarized in the table below:

| Determination of final weight | |
|---|---|
| average | 2314BLU01-C2 |
| Weight of 10 coated capsules - 1 | 8.5628 g |
| Weight of 10 coated capsules - 2 | 8.5736 g |
| Weight of 10 coated capsules - 3 | 8.5562 g |
| Mean final weight | 0.8564 g |

### 8.5.3.5 Process Mass Losses

Yield and reconciliation of coating process are presented below. Total losses from the manufacture of capsules are negligible.

| Batch | 2314BLU01-C2 | |
|---|---|---|
| Initial average tablet weight (PMI) | 837.7 | mg |
| Final average tablet weight (PMF) | 856.4 | mg |
| Weight gain (GM = (PMF − PM1)/PM1 × 100) | 2.23% | |
| Quantity of sprayed solution (QSP) | 34 | g |
| Dry matter solution concentration (MS) | 20.23% | |
| Dry matter sprayed (MSP = QSP * MS/100) | 6.88 | g |
| Mass of uncoated capsules (CR) | 221.88 | g |
| Masse capsules in destruction (CD) | N/A | |
| Mass of coated capsules (CP) | 207.76 | g |
| Sampling mass (PR) | 8.46 | g |
| Yield ((CP + PR)/(CR + MSP) × 100) | 94.5% | |
| Reconciliation ((CP + CD + PR)/(CR + MSP) × | 94.5% | |
| 100) | | |

### 8.6 Conclusion

It has been possible to coat Soctec® capsules with 14 mg Methylene Blue. The large amount of colouring in the coating suspension led to some nozzle clogging problems during coating of layer 1. This problem has been solved by filtering the coating suspension. A few stuck capsules were also observed, probably due to the large amount of colouring in the suspension and a slightly too fast spray rate.

For coating of layer 2, no issue occurred during the preparation of the coating suspensions, as well as the coating process.

RECORDING OF COATING PARAMETERS LAYER 1

| PARAMETER | RECORDING PARAMETERS | | | | | | | | |
|---|---|---|---|---|---|---|---|---|---|
| Step * | P | S | S | S | S | P | S | S | S |
| Time (min) | 3 | 5 | 10 | 15 | 19.2 | 1 | 20 | 25 | 30 |
| Air flow (m³/min) | 0.7 | 0.7 | 0.7 | 0.7 | 0.7 | 0.7 | 0.7 | 0.7 | 0.7 |
| Inlet air temperature (° C.) | 40 | 50 | 51 | 51 | 49 | 49 | 52 | 52 | 52 |
| Product temperature (° C.) | 41.7 | 31.8 | 32.5 | 46.4 | 37.6 | 42.3 | 30.6 | 32.9 | 33.5 |
| Outlet air temperature (° C.) | 35 | 34.8 | 34.2 | 38.9 | 34.2 | 33.2 | 33.4 | 35 | 35.9 |
| Drum speed (rpm) | 30 | 30 | 30 | 30 | 30 | 30 | 30 | 30 | 30 |
| Depression (mbar) | 4.5 | 4.5 | 4.5 | 4.5 | 4.5 | 4.5 | 4.5 | 4.5 | 4.5 |
| Spraying pressure (bar) | | 1 | 1 | 1 | 1 | | 1 | 1 | 1 |
| Atomization pressure (bar) | | 0.7 | 0.7 | 0.7 | 0.7 | | 0.7 | 0.7 | 0.7 |
| Pump speed (%) | | 35 | 35 | 35 | 35 | | 35 | 35 | 35 |
| Sprayed quantity (g) | | 15 | 33.3 | 36.1 | 39.5 | | 48.3 | 66.5 | 85 |
| Flow rate (g/min) | | 3 | 3.66 | 0.56 | 0.81 | | | 3.64 | 3.7 |
| Product appearance (C/NC) | C | C | C | C | C | C | C | C | C |
| Comments | N/A | N/A | N/A | clogging | clogging | preheating | N/A | N/A | 2 sticked |
| | | | | nozzle => | nozzle => | after | | | capsules |
| | | | | coating | coating | coating | | | |
| | | | | stop | stop | stop | | | |
| | | | | to unclog | to unclog | | | | |

| PARAMETER | RECORDING PARAMETERS | | | | | | | | |
|---|---|---|---|---|---|---|---|---|---|
| Step * | S | S | S | S | S | D | D | D | C |
| Time (min) | 35 | 40 | 45 | 50 | 53.8 | 10 | 20 | 30 | 17.4 |
| Air flow (m³/min) | 0.7 | 0.7 | 0.7 | 0.7 | 0.7 | 0.7 | 0.7 | 0.7 | 0.9 |
| Inlet air temperature (° C.) | 52 | 50 | 50 | 50 | 50 | 45 | 45 | 45 | OFF |
| Product temperature (° C.) | 34.7 | 33.5 | 34.1 | 33.5 | 33.5 | 50 | 50 | 50 | 30 |
| Outlet air temperature (° C.) | 36.7 | 36.4 | 36.6 | 37.2 | 37.4 | 41.5 | 41.5 | 41.5 | 28.5 |
| Drum speed (rpm) | 30 | 30 | 30 | 30 | 30 | 30 | 30 | 30 | 30 |
| Depression (mbar) | 4.5 | 5 | 5 | 5 | 5 | 5 | 5 | 5 | 7.3 |
| Spraying pressure (bar) | 1 | 1 | 1 | 1 | 1 | | | | |
| Atomization pressure (bar) | 0.7 | 0.7 | 0.7 | 0.7 | 0.7 | | | | |
| Pump speed (%) | 35 | 35 | 35 | 35 | 35 | | | | |
| Sprayed quantity (g) | 103.6 | 123.1 | 141.6 | 159.4 | 172.5 | | | | |
| Flow rate (g/min) | 3.72 | 3.9 | 3.7 | 3.56 | 3.45 | | | | |
| Product appearance (C/NC) | C | C | C | C | C | C | C | C | C |
| Comments | 4 sticked | 5 sticked | 6 sticked | 7 sticked | 8 sticked | 9 sticked | 10 sticked | 11 sticked | 12 sticked |
| | capsules | capsules | capsules | capsules | capsules | capsules | capsules | capsules | capsules |

RECORDING OF COATING PARAMETERS LAYER 2

| Batch number: | 2314BLU01− | | | | | | | |
|---|---|---|---|---|---|---|---|---|
| Step * | P | S | S | S | S | D | D | C |
| Time (min) | 3 | 5 | 10 | 15 | 18 | 10 | 20 | 9.3 |
| Air flow (m³/min) | 0.7 | 0.7 | 0.7 | 0.7 | 0.7 | 0.7 | 0.7 | 0.9 |
| Inlet air temperature (° C.) | 44 | 44 | 44 | 44 | 44 | 47 | 47 | OFF |
| Product temperature (° C.) | 41.2 | 32.3 | 34.1 | 34.7 | 35.3 | 48.8 | 48.8 | 30 |
| Outlet air temperature (° C.) | 34.4 | 32.6 | 33.5 | 34.1 | 36.6 | 40 | 40 | 27.8 |
| Drum speed (rpm) | 30 | 30 | 30 | 30 | 30 | 30 | 30 | 30 |
| Depression (mbar) | 4.3 | 4.3 | 4.3 | 4.3 | 4.3 | 4.8 | 4.8 | 7 |
| Spraying pressure (bar) | | 1 | 1 | 1 | 1 | | | |
| Atomization pressure (bar) | | 0.7 | 0.7 | 0.7 | 0.7 | | | |
| Pump speed (%) | | 25 | 25 | 25 | 25 | | | |
| Sprayed quantity (g) | | 8.5 | 17.7 | 27.6 | 34 | | | |
| Flow rate (g/min) | | 1.7 | 1.84 | 1.98 | 2.1 | | | |
| Product appearance (C/NC) | C | C | C | C | C | C | C | C |
| Comments | N/A | No | No | No | No | No | No | No |
| | | sticking | sticking | sticking | sticking | sticking | sticking | sticking |

* P = preheating // S = spraying // D = drying // C

### Example 9—Floating Gastro-Retentive LMTM Capsules

A gastroretentive capsule using SOCTEC® (Self Orienting Capsule Technology; see WO2012004231) but specifically adapted specifically to release between 5 and 20 mg of MT from LMTM in the stomach during the gastric residence time is prepared analogously to Example 8 used for MB.

For use with LMTM the Layer 1 composition in 8.5.1 includes LMTM in place of MB as API. It further includes a 2×mg ratio of ascorbic acid to maintain reduction. The amount of water is reduced correspondingly.

### Example 10—MTC Capsule Dissolution Studies

### a) Methods

Dissolution profile of the capsules prepared in Example 8 was determined and are presented hereafter. For batch, dissolution on 6 capsules and in one media (buffer pH 1.2, USP) were carried out. The test is carried out either by an automated dissolution system.

### 1 Equipment

Dissolution apparatus II (paddle)
Spectrophotometer
Cell (quartz): 0.5 cm
Filter 2.7 μm GF/D 25 mm Whatman—Ref. 1823-025

### 2 Reagents

| Component | N°CAS | Grade |
|---|---|---|
| Water | N/A | Purified Water |
| Potassium Chloride | 7447-40-7 | Merck, reference: P3911 |
| 2N hydrochloric acid | 7647-01-0 | 1.09063.1000 |
| Methylene blue | 122965-43-9 | M9140 |

### 3 Dissolution Parameters

| Parameters | Range | |
|---|---|---|
| Speed of rotation | 75 ± 4 | rpm |
| Test medium | pH 1.2 USP buffer | |
| Volume of test medium | 500 | mL |
| Temperature | 37.0 ± 0.5° | C. |
| Wavelength | 246 | nm |

| | |
|---|---|
| Reference | Test medium |

### 4 Solution Preparations

Alternative flask sizes and dilutions may be employed as long as the final concentration of the solution is maintained.

### 4.1 Dissolution Medium

Weigh and add 19 g of potassium chloride in water. Add 213 mL of 2N hydrochloric acid and complete to 5000 mL with purified water. Degas the dissolution medium for about 1 minute per liter.

### 4.2 Standard Solution

Weigh 16 mg of methylene blue and introduce in 500.0 mL volumetric flask. Complete to volume with dissolution medium. If necessary, sonicate to dissolve methylene blue.

The standard concentration is 32 mg/L of methylene blue.

### 5 Dissolution

Set up the dissolution apparatus using the parameters described hereafter. To each vessel, accurately add 500 mL of dissolution medium and allow to equilibrate to 37° C.±0.5° C. Weigh (form information) 6 capsules. Circulate the dissolution medium trough the cell and check the absence of leaks and bubbles. Record the temperature of each vessel. Perform the autozero with dissolution medium. Introduce the standard solution in a 0.5 cm quartz cell and place it in position 8 of the spectrophotometer. Start the dissolution with the software, using the specified parameters:
Read blanks on each vessel.
Introduce one capsule in each vessel.

### 6 Evaluation

Calculate the % of methylene blue dissolved for each point and each vessel, as follow:

Q% methylene blue dissolved per capsule=(Aₛₐₘₚₗₑ×W_{Std}×V)/(A_{Std}×V_{Std}×LC)×100

With:
Q(%)=Dissolution content
Aₛₐₘₚₗₑ: Absorbance of the sample
A_{Std}: Absorbance of the standard solution
W_{Std}: Weight of the standard solution
LC: Label claim (=16 mg)
V: Volume of medium in each vessel
V_{Std} Dilution volume of the standard solution

### b) Results—Profile Dissolution in pH 1.2 (Medium USP)

The first table shows calculation with capsule mass. In this table, we have calculated the dissolution content (Q %) as a function of capsule mass, to verify the impact of mass variation on the results

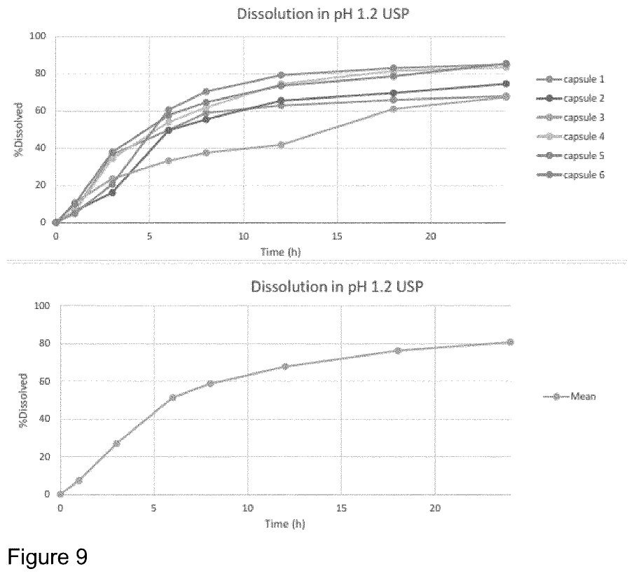

These results are summarised in FIG. 9.

$$Corrected Q \% Dissolved = ( \% dissolved above ) \times AW ) / WCaps$$

With:
AW: Average weight of capsule (mg)
Wcaps: Weight of the capsule (mg)

Q (%)

| time | capsule | capsule | capsule | capsule | capsule | capsule | |
|---|---|---|---|---|---|---|---|
| (h) | 1 | 2 | 3 | 4 | 5 | 6 | mean |
| 0 | 0 | 0 | 0 | 0 | 0.0 | 0 | 0 |
| 1 | 6 | 16 | 11 | 6 | 5 | 10 | 7 |
| 3 | 36 | 16 | 24 | 34 | 21 | 38 | 27 |
| 6 | 50 | 49 | 33 | 54 | 61 | 58 | 51 |
| 8 | 59 | 55 | 37 | 62 | 70 | 65 | 59 |
| 12 | 63 | 65 | 42 | 75 | 79 | 73 | 68 |
| 18 | 66 | 70 | 61 | 81 | 83 | 78 | 76 |
| 24 | 68 | 75 | 67 | 83 | 85 | 85 | 81 |

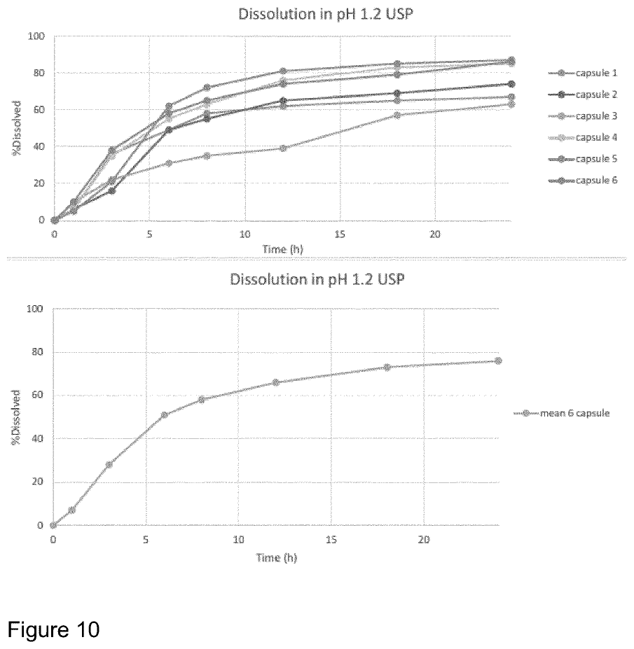

The second table shows calculation without capsule mass. These results are summarised in FIG. 10.

Q (%)

| Time | Capsule | Capsule | Capsule | Capsule | Capsule | Capsule | |
|---|---|---|---|---|---|---|---|
| (h) | 1 | 2 | 3 | 4 | 5 | 6 | mean |
| 0 | 0 | 0 | 0 | 0 | 0 | 0 | 0 |
| 1 | 6 | 6 | 10 | 6 | 5 | 10 | 7 |
| 3 | 36 | 16 | 22 | 35 | 21 | 38 | 28 |
| 6 | 49 | 49 | 31 | 55 | 62 | 58 | 51 |
| 8 | 58 | 55 | 35 | 63 | 72 | 65 | 58 |
| 12 | 62 | 65 | 39 | 76 | 81 | 74 | 66 |
| 18 | 65 | 69 | 57 | 83 | 85 | 79 | 73 |
| 24 | 67 | 74 | 63 | 85 | 87 | 86 | 76 |

As can be seen, the correction for capsule mass variation has no significant impact on the dissolution results.

These results demonstrate the effectiveness of a gastroretained, sustained-released MB composition according to the invention. Since the MB is released steadily over 24 hours, this demonstrated the feasibility of daily or less than daily (e.g. dosing every 2 or 3 days).

### REFERENCES FOR PROTEINS INVOLVED IN DISEASES OF PROTEIN AGGREGATION

Abrahamson, M., Jonsdottir, S., Olafsson, I. & Grubb, A. (1992) Hereditary cystatin C amyloid angiopathy identification of the disease-causing mutation and specific diagnosis by polymerase chain reaction based analysis. Human Genetics 89, 377-380.
Andersen, P. (2006) Amyotrophic lateral sclerosis associated with mutations in the CuZn superoxide dismutase gene. Current Neurology and Neuroscience Reports 6, 37-46.
Arai, T., Hasegawa, M., Nonoka, T., Kametani, F., Yamashita, M., Hosokawa, M., Niizato, K., Tsuchiya, K., Kobayashi, Z., Ikeda, K., Yoshida, M., Onaya, M., Fujishiro, H. &
Akiyama, H. (2010) Phosphorylated and cleaved TDP-43 in ALS, FTLD and other neurodegenerative disorders and in cellular models of TDP-43 proteinopathy. Neuropathology 30, 170-181.
Askanas, V., Engel, W. K. & Nogalska, A. (2009) Inclusion body myositis: a degenerative muscle disease associated with intra-muscle fiber multi-protein aggregates, proteasome inhibition, endoplasmic reticulum stress and decreased lysosomal degradation. Brain Pathology 19, 493-506.
Barmada, S. J., Skibinski, G., Korb, E., Rao, E. J., Wu, J. Y. & Finkbeiner, S. (2010) Cytoplasmic mislocalization of TDP-43 is toxic to neurons and enhanced by a mutation associated with familial amyotrophic lateral sclerosis. Journal of Neuroscience 30, 639-649.
Blair, I. P., Williams, K. L., Warraich, S. T., Durnall, J. C., Thoeng, A. D., Manavis, J., Blumbergs, P. C., Vucic, S., Kiernan, M. C. & Nicholson, G. A. (2010) FUS mutations in amyotrophic lateral sclerosis: clinical, pathological, neurophysiological and genetic analysis. Journal of Neurology Neurosurgery and Psychiatry 81, 639-645.
Booth, D. R., Sunde, M., Bellotti, V., Robinson, C. V., Hutchinson, W. L., Fraser, P. E., Hawkins, P. N., Dobson, C. M., Radford, S. E., Blake, C. C. F. & Pepys, M. B. (1997) Instability, unfolding and aggregation of human lysozyme variants underlying amyloid fibrillogenesis. Nature 385, 787-793.
Byrne, S., Walsh, C., Lynch, C., Bede, P., Elamin, M., Kenna, K., McLaughlin, R. & Hardiman, O. (2011) Rate of familial amyotrophic lateral sclerosis: a systematic review and meta-analysis. Journal of Neurology, Neurosurgery & Psychiatry 82, 623-627.
Carrell, R. W. & Gooptu, B. (1998) Conformational changes and disease—serpins, prions and Alzheimer's. Current Opinion in Structural Biology 8, 799-809.
Chen-Plotkin, A. S., Lee, V. M. Y. & Trojanowski, J. Q. (2010) TAR DNA-binding protein 43 in neurodegenerative disease. Nature Reviews Neurology 6, 211-220.
Chiti, F., Webster, P., Taddei, N., Clark, A., Stafani, M., Ramponi, G. & Dobson, C. (1999) Designing conditions for in vitro formation of amyloid protofilaments and fibrils. Proceedings of the National Academy of Sciences, USA 96, 3590-3594.
Cox, L. E., Ferraiuolo, L., Goodall, E. F., Heath, P. R., Higginbottom, A., Mortiboys, H., Hollinger, H. C., Hartley, J. A., Brockington, A., Burness, C. E., Morrison, K. E., Wharton, S. B., Grierson, A. J., Ince, P. G., Kirby, J. & Shaw, P. J. (2010) Mutations in CHMP2B in lower motor neuron predominant amyotrophic lateral sclerosis (ALS). PLOS One 5, e9872.
Czech, C., Tremp, G. & Pradier, L. (2000) Presenilins and Alzheimer's disease: biological functions and pathogenic mechanisms. Progress in Neurobiology 60, 363-384.
Davis, R. L., Shrimpton, A. E., Holohan, P. D., Bradshaw, C., Feiglin, D., Collins, G. H., Sonderegger, P., Kinter, J., Becker, L. M., Lacbawan, F., Krasnewich, D., Muenke, M., Lawrence, D. A., Yerby, M. S., Shaw, C.-M., Gooptu, B., Elliott, P. R., Finch, J. T., Carrell, R. W. & Lomas, D. A. (1999) Familial dementia caused by polymerization of mutant neuroserpin. Nature 401, 376-379.
DiFiglia, M., Sapp, E., Chase, K. O., Davies, S. W., Bates, G. P., Vonsattel, J. P. & Aronin, N. (1997) Aggregation of huntingtin in neuronal intranuclear inclusions and dystrophic neurites in brain. Science 277, 1990-1993.
Dische, F. E., Wernstedt, C., Westermark, G. T., Westermark, P., Pepys, M. B., Rennie, J. A., Gilbey, S. G. & Watkins, P. J. (1988) Insulin as an amyloid-fibril protein at sites of repeated insulin injections in a diabetic patient. Diabetologia 31, 158-161.
Elden, A. C., Kim, H.-J., Hart, M. P., Chen-Plotkin, A. S., Johnson, B. S., Fang, X., Armakola, M., Geser, F., Greene, R., Lu, M. M., Padmanabhan, A., Clay-Falcone, D., McCluskey, L., Elman, L., Juhr, D., Gruber, P. J., Rub, U., Auburger, G., Trojanowski, J. Q., Lee, V. M. Y., Van Deerlin, V. M., Bonini, N. M. & Gitler, A. D. (2010) Ataxin-2 intermediate-length polyglutamine expansions are associated with increased risk for ALS. Nature 466, 1069-1075.
Finsterer, J (2009) Mitochondrial disorders, cognitive impairment and dementia. J. Neurol. Sci. 283:143-148
Gasset, M., Bladwin, M. A., Lloyd, D. H., abriel, J.-M., Holtzman, D. M., Cohen, F. E., Fletterick, R. & Prusiner, S. B. (1992) Predicted a-helical region of the prion protein when synthesized as peptides form amyloid. Proceedings of the National Academy of Sciences, USA 89, 10940-10944.
Gendron, T. F., Josephs, K. A. & Petrucelli, L. (2010) Review: Transactive response DNA-binding protein 43 (TDP-43): mechanisms of neurodegeneration. Neuropathology and Applied Neurobiology 36, 97-112.
Geser, F., Lee, V. M.-Y. & Trojanowski, J. Q. (2010) Amyotrophic lateral sclerosis and frontotemporal lobar degeneration: A spectrum of TDP-43 proteinopathies. Neuropathology 30, 103-112.
Gitcho, M. A., Baloh, R. H., Chakraverty, S., Mayo, K., Norton, J. B., Levitch, D., Hatanpaa, K. J., White, C. L., III, Bigio, E. H., Caselli, R., Baker, M., AI-Lozi, M. T., Morris, J. C., Pestronk, A., Rademakers, R., Goate, A. M. & Cairns, N. J. (2008) TDP-43 A315T mutation in familial motor neuron disease. Annals of Neurology 63, 535-538.
Glenner, G. G. & Wong, C. W. (1984) Alzheimer's disease: initial report of the purification and characterisation of a novel cerebrovascular amyloid protein. Biochemical and Biophysical Research Communications 120, 885-890.
Goate, A., Chartier-Harlin, M.-C., Mullan, M., Brown, J., Crawford, F., Fidani, L., Giuffra, L., Haynes, A., Irving, N., James, L., Mant, R., Newton, P., Rooke, K., Roques, P., Talbot, C., Pericak-Vance, M., Roses, A., Williamson, R., Rossor, M., Owen, M. & Hardy, J. (1991) Segregation of a missense mutation in the amyloid precursor protein gene with familial Alzheimer's disease. Nature 349, 704-706.
Gorevic, P. D., Casey, T. T., Stone, W. J., DiRaimondo, C. R., Prelli, F. C. & Frangione, B. (1985) b-2 Microglobulin is an amyloidogenic protein in man. Journal of Clinical Investigation 76, 2425-2429.
Gustavsson, A., Engström, U. & Westermark, P. (1991) Normal transthyretin and synthetic transthyretin fragments form amyloid-like fibrils in vitro. Biochemical and Biophysical Research Communications 175, 1159-1164.
Higashi, S., Tsuchiya, Y., Araki, T., Wada, K. & Kabuta, T. (2010) TDP-43 physically interacts with amyotrophic lateral sclerosis-linked mutant CuZn superoxide dismutase. Neurochemistry International 57, 906-913.
Hutton, M., Lendon, C., Rizzu, P., Baker, M., Froelich, S., Houlden, H., Pickering-Brown, S., Chakraverty, S., Isaacs, A., Grover, A., Hackett, J., Adamson, J., Lincoln, S., Dickson, D., Davies, P., Petersen, R. C., Stevens, M., de Graaf, E., Wauters, E., van Baren, J., Hillebrand, M., Joosse, M., Kwon, J. M., Nowotny, P., Che, L. K., Norton, J., Morris, J. C., Reed, L. A., Trojanowski, J. Q., Basun, H., Lannfelt, L., Neystat, M., Fahn, S., Dark, F., Tannenberg, T., Dodd, P. R., Hayward, N., Kwok, J. B. J., Schofield, P. R., Andreadis, A., Snowden, J., Craufurd, D., Neary, D., Owen, F., Oostra, B. A., Hardy, J., Goate, A., van Swieten, J., Mann, D., Lynch, T. & Heutink, P. (1998) Association of missense and 5′-splice-site mutations in tau with the inherited dementia FTDP-17. Nature 393, 702-705.
Igaz, L. M., Kwong, L. K., Chen-Plotkin, A., Winton, M. J., Unger, T. L., Xu, Y., Neumann, M., Trojanowski, J. Q. & Lee, V. M. Y. (2009) Expression of TDP-43 C-terminal fragments in vitro recapitulates pathological features of TDP-43 proteinopathies. Journal of Biological Chemistry 284, 8516-8524.
Jinwal, U K, Miyata, Y, Koren, J, III, Jones, J R, Trotter, J H et al. (2009) Chemical manipulation of Hsp70 ATPase activity regulates tau stability. J. Neurosci. 29:12079-12088
Johansson, B., Wernstedt, C. & Westermark, P. (1987) Atrial natriuretic peptide deposited as atrial amyloid fibrils. Biochemical and Biophysical Research Communications 148, 1087-1092.
Johnson, B. S., McCaffery, J. M., Lindquist, S. & Gitler, A. D. (2008) A yeast TDP-43 proteinopathy model: Exploring the molecular determinants of TDP-43 aggregation and cellular toxicity. Proceedings of the National Academy of Sciences 105, 6439-6444.
Johnson, B. S., Snead, D., Lee, J. J., McCaffery, J. M., Shorter, J. & Gitler, A. D. (2009) TDP-43 is intrinsically aggregation-prone, and amyotrophic lateral sclerosis-linked mutations accelerate aggregation and increase toxicity. Journal of Biological Chemistry 284, 20329-20339.
Johnson, J. O., Mandrioli, J., Benatar, M., Abramzon, Y., Van Deerlin, V. M., Trojanowski, J. Q., Gibbs, J. R., Brunetti, M., Gronka, S., Wuu, J., Ding, J., McCluskey, L., Martinez-Lage, M., Falcone, D., Hernandez, D. G., Arepalli, S., Chong, S., Schymick, J. C., Rothstein, J., Landi, F., Wang, Y.-D., Calvo, A., Mora, G., Sabatelli, M., Monsurro, M. R., Battistini, S., Salvi, F., Spataro, R., Sola, P., Borghero, G., Galassi, G., Scholz, S. W., Taylor, J. P., Restagno, G., Chió, A. & Traynor, B. J. (2010) Exome sequencing reveals VCP mutations as a cause of familial ALS. Neuron 68, 857-864.
Kabashi, E., Lin, L., Tradewell, M. L., Dion, P. A., Bercier, V., Bourgouin, P., Rochefort, D., Bel Hadj, S., Durham, H. D., Velde, C. V., Rouleau, G. A. & Drapeau, P. (2010) Gain and loss of function of ALS-related mutations of TARDBP (TDP-43) cause motor deficits in vivo. Human Molecular Genetics 19, 671-683.
Kabashi, E., Valdmanis, P. N., Dion, P., Spiegelman, D., McConkey, B. J., Velde, C. V., Bouchard, J.-P., Lacomblez, L., Pochigaeva, K., Salachas, F., Pradat, P.-F., Camu, W., Meininger, V., Dupre, N. & Rouleau, G. A. (2008) TARDBP mutations in individuals with sporadic and familial amyotrophic lateral sclerosis. Nature Genetics 40, 572-574.
Ling, S.-C., Albuquerque, C. P., Han, J. S., Lagier-Tourenne, C., Tokunaga, S., Zhou, H. & Cleveland, D. W. (2010) ALS-associated mutations in TDP-43 increase its stability and promote TDP-43 complexes with FUS/TLS. Proceedings of the National Academy of Sciences 107, 13318-13323.
Lomas, D. A., Evans, D. L., Finch, J. T. & Carrell, R. W. (1992) The mechanism of Z al-antitrypsin accumulation in the liver. Nature 357, 605-607.
Love, S., Bridges, L. R. & Case, C. P. (1995) Neurofibrillary tangles in Niemann-Pick disease type C. Brain 118, 119-129.
Mackenzie, I. R. A., Bigio, E. H., Ince, P. G., Geser, F., Neumann, M., Cairns, N. J., Kwong, L. K., Forman, M. S., Ravits, J., Stewart, H., Eisen, A., McClusky, L., Kretzschmar, H. A., Monoranu, C. M., Highley, J. R., Kirby, J., Siddique, T., Shaw, P. J., Lee, V. M. Y. & Trojanowski, J. Q. (2007) Pathological TDP-43 distinguishes sporadic amyotrophic lateral sclerosis from amyotrophic lateral sclerosis with SOD1 mutations. Annals of Neurology 61, 427-434.
Mackenzie, I. R. A., Rademakers, R. & Neumann, M. (2010) TDP-43 and FUS in amyotrophic lateral sclerosis and frontotemporal dementia. The Lancet Neurology 9, 995-1007.
Maury, C. P. & Baumann, M. (1990) Isolation and characterization of cardiac amyloid in familial amyloid polyneuropathy type IV (Finnish): relation of the amyloid protein to variant gelsolin. Biochimica et Biophysica Acta 1096, 84-86.
Neary, D., Snowden, J. S., Gustafson, L., Passant, U., Stuss, D., Black, S., Freedman, M., Kertesz, A., Robert, P. H., Albert, M., Boone, K., Miller, B. L., Cummings, J. & Benson, D. F. (1998) Frontotemporal lobar degeneration: a consensus on clinical diagnostic criteria. Neurology 51, 1546-1554.
Neumann, M. (2009) Molecular neuropathology of TDP-43 proteinopathies. International Journal of Molecular Sciences 10, 232-246.
Neumann, M., Sampathu, D. M., Kwong, L. K., Truax, A. C., Micsenyi, M. C., Chou, T. T., Bruce, J., Schuck, T., Grossman, M., Clark, C. M., McCluskey, L. F., Miller, B. L., Masliah, E., Mackenzie, I. R., Feldman, H., Feiden, W., Kretzschmar, H. A., Trojanowski, J. Q. & Lee, V. M. Y. (2006) Ubiquitinated TDP-43 in frontotemporal lobar degeneration and amyotrophic lateral sclerosis. Science 314, 130-133.
Nonaka, T., Kametani, F., Arai, T., Akiyama, H. & Hasegawa, M. (2009) Truncation and pathogenic mutations facilitate the formation of intracellular aggregates of TDP-43. Human Molecular Genetics 18, 3353-3364.
Ohmi, K., Kudo, L. C., Ryazantsev, S., Zhao, H.-Z., Karsten, S. L. & Neufeld, E. F. (2009) Sanfilippo syndrome type B, a lysosomal storage disease, is also a tauopathy. Proceedings of the National Academy of Sciences 106, 8332-8337.
Orr, H. T. & Zoghbi, H. Y. (2007) Trinucleotide repeat disorders. Annual Review of Neuroscience 30, 575-621.
Paulson, H. L. (1999) Human genetics '99: trinucleotide repeats. American Journal of Human Genetics 64, 339-345.
Pepys, M. B., Hawkins, P. N., Booth, D. R., Vigushin, D. M., Tennent, G. A., Soutar, A. K., Totty, N., Nguyen, O., Blake, C. C. F., Terry, C. J., Feest, T. G., Zalin, A. M. & Hsuan, J. J. (1993) Human lysozyme gene mutations cause hereditary systemic amyloidosis. Nature 362, 553-557.
Polymeropoulos, M. H., Lavedan, C., Leroy, E., Ide, S. E., Dehejia, A., Dutra, A., Pike, B., Root, H., Rubenstein, J., Boyer, R., Stenroos, E. S., Chandrasekharappa, S., Athanassiadou, A., Papaetropoulos, T., Johnson, W. G., Lazzarini, A. M., Duvoisin, R. C., Di Iorio, G., Golbe, L. I. & Nussbaum, R. L. (1997) Mutation in the a-synuclein gene identified in families with Parkinson's disease. Science 276, 2045-2047.
Prusiner, S. B., Scott, M. R., DeArmond, S. J. & Cohen, F. E. (1998) Prion protein biology. Cell 93, 337-348.
Seetharaman, S. V., Prudencio, M., Karch, C., Holloway, S. P., Borchelt, D. R. & Hart, P. J. (2009) Immature copper-zinc superoxide dismutase and familial amyotrophic lateral sclerosis. Experimental Biology and Medicine 234, 1140-1154.
Seilhean, D., Cazeneuve, C., Thuries, V., Russaouen, O., Millecamps, S., Salachas, F., Meininger, V., LeGuern, E. & Duyckaerts, C. (2009) Accumulation of TDP-43 and α-actin in an amyotrophic lateral sclerosis patient with the K171 ANG mutation Acta Neuropathologica 118, 561-573.
Shibata, N., Hirano, A., Kobayashi, M., Siddique, T., Deng, H. X., Hung, W. Y., Kato, T. & Asayama, K. (1996) Intense superoxide dismutase-1 immunoreactivity in intracytoplasmic hyaline inclusions of familial amyotrophic lateral sclerosis with posterior column involvement. Journal of Neuropathology and Experimental Neurology 55, 481-490.
Sletten, K., Westermark, P. & Natvig, J. B. (1976) Characterization of amyloid fibril proteins from medullary carcinoma of the thyroid. Journal of Experimental Medicine 143, 993-998.
Spillantini, M. G., Crowther, R. A., Jakes, R., Hasegawa, M. & Goedert, M. (1998) a-Synuclein in filamentous inclusions of Lewy bodies from Parkinson's disease and dementia with Lewy bodies. Proceedings of the National Academy of Sciences, USA 95, 6469-6473.
Sreedharan, J., Blair, I. P., Tripathi, V. B., Hu, X., Vance, C., Rogelj, B., Ackerley, S., Durnall, J. C., Williams, K. L., Buratti, E., Baralle, F., de Belleroche, J., Mitchell, J. D., Leigh, P. N., AI-Chalabi, A., Miller, C. C., Nicholson, G. & Shaw, C. E. (2008) TDP-43 mutations in familial and sporadic amyotrophic lateral sclerosis. Science 319, 1668-1672.
Uemichi, T., Liuepnicks, J. j. & Benson, M. D. (1994) Hereditary renal amyloidosis with a novel variant fibrinogen. Journal of Clinical Investigation 93, 731-736. van Bebber, F., Paquet, D., Hruscha, A., Schmid, B. & Haass, C. (2010) Methylene blue fails to inhibit Tau and polyglutamine protein dependent toxicity in zebrafish. Neurobiology of Disease 39, 265-271.
Vance, C., Rogelj, B., Hortobagyi, T., De Vos, K. J., Nishimura, A. L., Sreedharan, J., Hu, X., Smith, B., Ruddy, D., Wright, P., Ganesalingam, J., Williams, K. L., Tripathi, V., Al-Saraj, S., AI-Chalabi, A., Leigh, P. N., Blair, I. P., Nicholson, G., de Belleroche, J., Gallo, J.-M., Miller, C. C. & Shaw, C. E. (2009) Mutations in FUS, an RNA processing protein, cause familial amyotrophic lateral sclerosis type 6. Science 323, 1208-1211.
Westermark, P., Engstrom, U., Johnson, K. H., Westermark, G. T. & Betsholtz, C. (1990) Islet amyloid polypeptide: pinpointing amino acid residues linked to amyloid fibril formation. Proceedings of the National Academy of Sciences, USA 87, 5036-5040.
Westermark, P., Johnson, K. H. & Pitkanen, P. (1985) Systemic amyloidosis: A review with emphasis on pathogenesis. Applied Physiology 3, 55-68.
Westermark, P., Johnson, K. H., O'Brien, T. D. & Betsholtz, C. (1992) Islet amyloid polypeptide—a novel controversy in diabetes research. Diabetologia 35, 297-303.
Wijesekera, L. & Leigh, P. N. (2009) Amyotrophic lateral sclerosis. Orphanet Journal of Rare Diseases 4, 3.
Wischik, C. M., Novak, M., Thogersen, H. C., Edwards, P. C., Runswick, M. J., Jakes, R., Walker, J. E., Milstein, C., M., R. & Klug, A. (1988) Isolation of a fragment of tau derived from the core of the paired helical filament of Alzheimer's disease. Proceedings of the National Academy of Sciences, USA 85, 4506-4510.
Yamashita, M., Nonaka, T., Arai, T., Kametani, F., Buchman, V. L., Ninkina, N., Bachurin, S. O., Akiyama, H., Goedert, M. & Hasegawa, M. (2009) Methylene blue and dimebon inhibit aggregation of TDP-43 in cellular models. FEBS Letters 583, 2419-2424.
Zhang, Y.-J., Xu, Y.-F., Cook, C., Gendron, T. F., Roettges, P., Link, C. D., Lin, W.-L., Tong, J., Castanedes-Casey, M., Ash, P., Gass, J., Rangachari, V., Buratti, E., Baralle, F., Golde, T. E., Dickson, D. W. & Petrucelli, L. (2009) Aberrant cleavage of TDP-43 enhances aggregation and cellular toxicity. Proceedings of the National Academy of Sciences 106, 7607-76.

1. A pharmaceutical composition which is a film-coated solid tablet comprising a diaminophenothiazine (DAPTZ) compound as active ingredient in a gastro-retentive platform,
wherein the DAPTZ compound is selected from an oxidized methylthioninium (MT⁺) compound or a leucomethylthioninium (LMT) compound or a combination thereof, wherein the formulation comprises a rapid release matrix including a hydrophilic macromolecule and is adapted to float in the stomach,
where in the formulation further comprises one or more other accompanying active ingredients, additives, excipients, diluents, binders, lubricants, disintegrators, fillers, stabilizers, surfactants, antioxidants, or combinations thereof.

2. A composition as claimed in claim 1, wherein the composition has a floating time of at least 120 seconds, more preferably at least 3, 4 or 5 minutes when placed on potable water.

3. A composition as claimed in claim 1 or claim 2, wherein the composition has a disintegration time of less than 5 minutes, preferably less than 4 minutes, or less than 3 minutes.

4. A composition as claimed in any one of claims 1 to 3, wherein the composition has a disintegration time in 50 to 200% of the floating time.

5. A composition as claimed in any one of claims 1 to 4, wherein the amount of MT⁺ or LMT in the composition is equal to or between 1 and 30 mg, more preferably about 1 to 20, about 1 to 10, about 2 to 6 mg, about 2 to 5 mg, more preferably about or equal to 4 mg.

6. A composition as claimed in any one of claims 1 to 5, wherein the composition is generally circular with a diameter of about 4 to 6 mm, optionally about 5 mm, and a thickness of 2 to 3 mm, optionally 2.2 to 2.6 mm.

7. A composition as claimed in any one of claims 1 to 6, wherein the composition has a hardness of 3 to 9 kp, preferably about 6 to 7 kp.

8. A composition as claimed in any one of claims 1 to 7, wherein the composition has a total weight of equal to or between 30 and 60 mg, optionally 40 and 60 mg, 50 and 60 mg, optionally about 53 mg, 54 mg, 55 mg, 56 mg, 56 mg, 58 mg, 59 mg, 60 mg.

9. A composition as claimed in any one of claims 1 to 8, wherein the composition has a bulk density of less than 1.5 g/cm³, optionally about 0.8 to 1.5 g/cm³, optionally about 1.0, 1.1, 1.2, 1.3 or 1.4, e.g. about 1.2, 1.3 or 1.4 g/cm³.

10. A composition as claimed in any one of claims 1 to 9, wherein the composition comprises a diluent which is optionally mannitol and/or microcrystalline cellulose.

11. A composition as claimed in any one of claims 1 to 10, wherein the composition comprises a disintegrant which is optionally crospovidone.

12. A composition as claimed in any one of claims 1 to 11, wherein the composition comprises a lubricant which is optionally magnesium stearate.

13. A composition as claimed in any one of claims 1 to 12, wherein the composition has a friability of less 1%.

14. A composition as claimed in any one of claims 1 to 13, wherein the hydrophilic macromolecule is a hydrophilic polymer which is Poly(vinyl alcohol) part-hydrolysed (PVA) which is present in the film coating on the tablet.

15. A composition as claimed in claim 14, wherein the coating further comprises one or more of: talc, titanium dioxide, macrogel 3350, lecithin, colouring, which is optionally FD&C blue #2/indigo carmine aluminium lake (dialuminum;2-(3-hydroxy-5-sulfonato-1H-indol-2-yl)-3-oxoindole-5-sulfonate).

16. A composition as claimed in claim 14 or claim 15 wherein the film coating is an Opadry® II coating.

17. A composition as claimed in any one of claims 14 to 16, wherein the coating is 5 to 8% of the total tablet weight.

18. A process for the manufacture of the pharmaceutical composition according to any one of claims 1 to 17, which process comprises:
(i) compression or granulation of the DAPTZ compound with the one or more other accompanying active ingredients, additives, excipients, diluents, binders, lubricants, disintegrants, fillers, stabilizers, surfactants, antioxidants, if present;
(ii) applying the film coating to the tablets,
optionally wherein the step of applying a film coating is carried out by spray-coating the tablet core in a coating machine, wherein the coating machine and cores are optionally pre-heated to 42-52° C., and adjusting temperature of inlet air so the exhaust temperature is maintained between 42-52° C.

19. An oral pharmaceutical composition comprising an elongate hollow cylindrical capsule which is impermeable to gastric fluid and having a fill volume inside containing a weighting agent which is retained at one end of the fill volume, the capsule being buoyant and self-orientating in an aqueous fluid such that it floats in the aqueous fluid with its long axis perpendicular to the surface of the aqueous fluid,
wherein the capsule is coated with a first inner sustained release layer comprising a diaminophenothiazine (DAPTZ) compound as active ingredient,
and wherein the first layer is coated with a second outer layer not including an active ingredient,
wherein the DAPTZ compound is selected from an oxidized methylthioninium (MT) compound or a leucomethylthioninium (LMT) compound or a combination thereof,
wherein the first and second layers each comprise at least one hydrophilic polymer and at least one hydrophobic polymer, plus optionally one or more lubricants, gluidants or plasticisers.

20. A composition as claimed in claim 19, wherein the first and second layers are partly or wholly insoluble.

21. A composition as claimed in claim 19 or claim 20, wherein the first layer makes up to 5 to 20% of the total weight of the composition.

22. A composition as claimed in any one of claims 19 to 21, wherein the second layer makes up about 1 to 5% of the total weight of the final dosage form.

23. A composition as claimed in any one of claims 19 to 22, wherein the hydrophilic polymer and the hydrophobic polymer are both water-insoluble acrylic copolymers, which are optionally acrylate/ammonium methacrylate copolymers.

24. A composition as claimed in any one of claims 19 to 23, wherein the hydrophobic polymer is Eudragit® RS 30D.

25. A composition as claimed in any one of claims 19 to 24, wherein the hydrophilic polymer is Eudragit® RL 30D.

26. A composition as claimed in any one of claims 19 to 25, wherein the weight ratio of first layer hydrophobic polymer to hydrophilic polymer is about 4:1.

27. A composition as claimed in any one of claims 19 to 26, wherein the weight ratio of second layer hydrophobic polymer to hydrophilic polymer is about 1:4.

28. A composition as claimed in any one of claims 19 to 27, wherein each layer further comprises
(a) a plasticizer, which is optionally a citrate ester, which is optionally triethyl citrate, and/or,
(b) a lubricant or gluidant, which is optionally talc.

29. A composition as claimed in any one of claims 19 to 28, wherein the amount of MT⁺ or LMT in the composition is equal to or between 10 and 120 mg.

30. A composition as claimed in any one of claims 19 to 29, wherein the capsule has a diameter of about 8 to 9 mm and a locked length of about 23 to 24 mm, and optionally wherein the weighting agent comprises barium sulfate.

31. A composition as claimed in any one of claims 19 to 30, wherein the composition releases less than 10% of the DAPTZ compound in pH 1.2 USP buffer within 1 hour of adding to the buffer, and more than 50% within 8 hours.

32. A composition as claimed in any one of claims 19 to 30, wherein the composition releases less than 10% of the DAPTZ compound in pH 1.2 USP buffer within 1, 2, 3, 4 or 8 hours of adding to the buffer, and more than 80% within 24, 48 or 72 hours.

33. A process for the manufacture of the pharmaceutical composition according to any one of claims 19 to 32, which process comprises:
(i) providing the capsule, and pre-heating it to obtain a product temperature of 35-40° C.;
(ii) spraying the capsule with a filtered first layer coating suspension until the required weight gain is achieved;
(iii) drying the coated capsules for 30 minutes to form a sustained polymeric first layer film coating,
(iv) optionally waiting for up to 24 hours;
(v) pre-heating the coated capsules to obtain a product temperature of about 40° C.;
(vi) spraying the coated capsule with a second layer coating suspension until the required weight gain is achieved;
(vii) drying the coated capsules for 20 minutes to form a sustained polymeric second layer film coating,
(viii) cooling of the capsules until a product temperature of 30° C. was obtained.

34. A process as claimed in claim 33 wherein each coating suspension is an aqueous suspension, optionally as shown in Table 3.

35. A pharmaceutical composition obtainable by the process of any one of claims 18, 33 or 34.

36. A composition or process as claimed in any one of claims 1 to 35, wherein the DAPTZ compound is a salt of either: 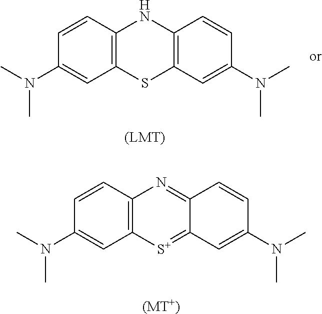
or a hydrate or solvate thereof.

37. A composition or process as claimed in claim 36, wherein the composition comprises a mixture of LMT and MT⁺ containing compounds.

38. A composition or process as claimed in claim 36, wherein the DAPTZ compound is an LMT compound.

39. A composition or process as claimed in claim 38 wherein the DAPTZ compound is an LMTX compound of the following formula: 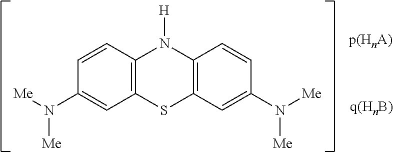
wherein each of HₙA and HₙB (where present) are protic acids which may be the same or different,
and wherein p=1 or 2; q=0 or 1; n=1 or 2; (p+q)×n=2.

40. A composition or process as claimed in claim 39 wherein the DAPTZ compound has the following formula, where HA and HB are different mono-protic acids:

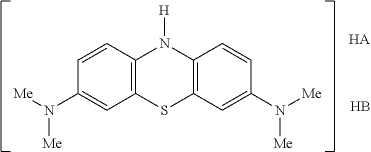

41. A composition or process as claimed in claim 39 wherein the DAPTZ compound has the following formula: 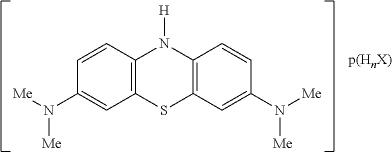
wherein each of HₙX is a protic acid.

42. A composition or process as claimed in claim 39 wherein the DAPTZ compound has the following formula and H₂A is a di-protic acid:

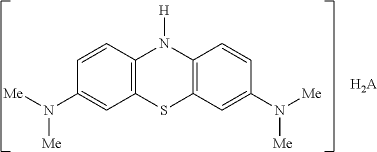

43. A composition or process as claimed in claim 39 wherein the DAPTZ compound has the following formula and is a bis-monoprotic acid:

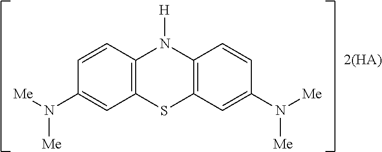

44. A composition or process as claimed in any one of claims 39 to 43 wherein the or each protic acid is an inorganic acid.

45. A composition or process as claimed in claim 44 wherein each protic acid is a hydrohalide acid.

46. A composition or process as claimed in claim 44 or claim 45 wherein the or each protic acid is selected from HCl; HBr; HNO₃; H₂SO₄.

47. A composition or process as claimed in any one of claims 39 to 43 wherein the or each protic acid is an organic acid.

48. A composition or process as claimed in claim 44 or claim 47 wherein the or each protic acid is selected from H₂CO₃; CH₃COOH; methanesulfonic acid, 1,2-ethanedisulfonic acid, ethanesulfonic acid, naphthalenedisulfonic acid, p-toluenesulfonic acid.

49. A composition or process as claimed in claim 48 wherein the DAPTZ compound is LMTM:

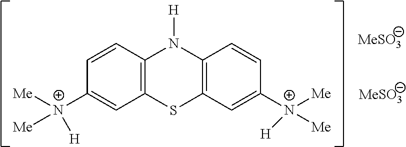

50. A composition or process as claimed in claim 48 wherein the DAPTZ compound is selected from the list consisting of:

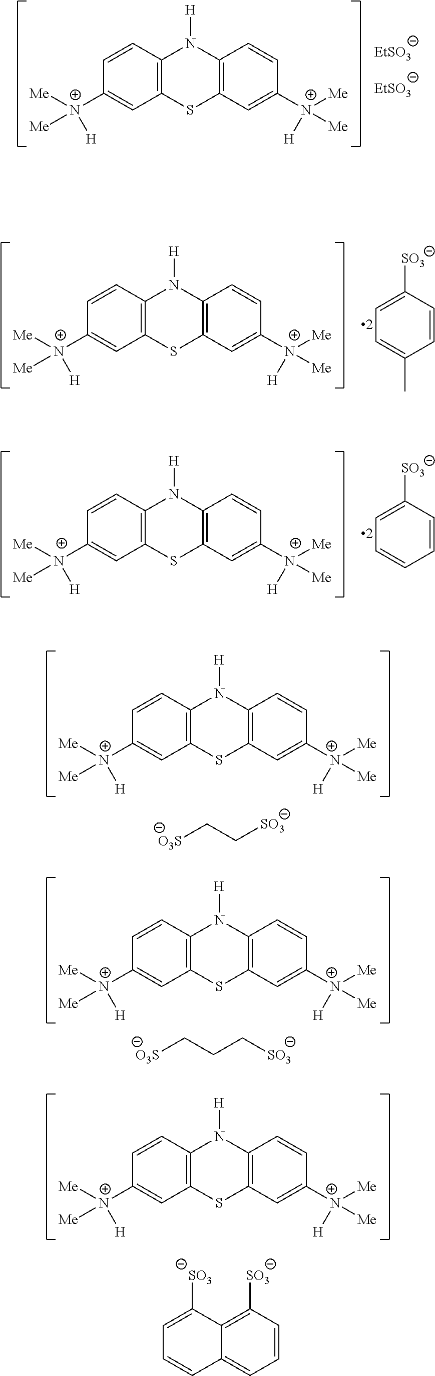

51. A composition or process as claimed in claim 36 wherein the DAPTZ compound is an MT⁺ salt having the formula or being a hydrate, solvate, or mixed salt thereof: 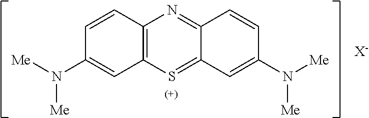
where X⁻ is an anionic counter ion.

52. A composition or process as claimed in claim 51 wherein the DAPTZ compound is MTC.

53. A composition or process as claimed in claim 52 wherein the MTC is MTC polymorph A pentahydrate.

54. A composition or process as claimed in any one of claims 51 to 53 wherein the MTC is characterised by a purity of greater than 98%.

55. A composition or process as claimed in any one of claims 51 to 54, wherein the MTC is characterised by a purity of greater than 98% and one or more of the following:
(i) less than 1% Azure B as impurity;
(ii) less than 0.13% MVB (Methylene Violet Bernstein) as impurity;
(iii) less than 0.15% Azure A as impurity;
(iv) less than 0.15% Azure C as impurity; or
(v) an elementals purity better than less than 100 μg/g Aluminium (AI); less than 1 μg/g Cadmium (Cd); less than 100 μg/g Chromium (Cr); less than 300 μg/g Copper (Cu); less than 10 μg/g Tin (Sn); less than 200 μg/g Iron (Fe); less than 10 μg/g Manganese (Mn); less than 1 μg/g Mercury (Hg); less than 10 μg/g Molybdenum (Mo); less than 10 μg/g Nickel (Ni); less than 10 μg/g Lead (Pb); and less than 100 μg/g Zinc (Zn).

56. A composition or process as claimed in any one of claims 51 to 55, wherein the MTC is characterised by a purity of greater than 98% and less than 1% Azure B as impurity.

57. A composition or process as claimed in any one of claims 51 to 56, wherein the MTC is characterised by a purity of greater than 98% and an elementals purity better than less than 100 μg/g Aluminium (AI); less than 1 μg/g Cadmium (Cd); less than 100 μg/g Chromium (Cr); less than 300 μg/g Copper (Cu); less than 10 μg/g Tin (Sn); less than 200 μg/g Iron (Fe); less than 10 μg/g Manganese (Mn); less than 1 μg/g Mercury (Hg); less than 10 μg/g Molybdenum (Mo); less than 10 μg/g Nickel (Ni); less than 10 μg/g Lead (Pb); and less than 100 μg/g Zinc (Zn).

58. A composition or process as claimed in any one of claims 51 to 57, wherein the MTC is characterised by:
(i) at least 98% purity
(i) less than 1% Azure B as impurity; and
(ii) an elementals purity better than the European Pharmacopeia limits of less than 100 μg/g Aluminium (AI); less than 1 μg/g Cadmium (Cd); less than 100 μg/g Chromium (Cr); less than 300 μg/g Copper (Cu); less than 10 μg/g Tin (Sn); less than 200 μg/g Iron (Fe); less than 10 μg/g Manganese (Mn); less than 1 μg/g Mercury (Hg); less than 10 μg/g Molybdenum (Mo); less than 10 μg/g Nickel (Ni); less than 10 μg/g Lead (Pb); and less than 100 μg/g Zinc (Zn).

59. A composition or process as claimed in claim 52 wherein the DAPTZ compound is selected from: MTC·0.5ZnCl₂; MTI; MTI·HI; MT·NO₃.

60. A method of treatment of a disease in a human subject, which method comprises administering to said subject a composition as claimed in any one of claims 1 to 17, 19 to 32, or 35 to 59.

61. A method as claimed in claim 60 wherein the disease is a neurodegenerative disorder of protein aggregation.

62. A method as claimed in claim 60 or claim 61, wherein said administration provides an amount of MT to the subject that corresponds to an average of between 0.05 mg and 30 mg MT per day, preferably between 0.1 mg and 20 mg MT per day, for a treatment period of at least 1 month.

63. A method as claimed in any one of claims 60 to 62 wherein the subject is a human who has been diagnosed as having said disorder, or wherein said method comprises making said diagnosis.

64. A method of prophylactic treatment of a neurodegenerative disorder of protein aggregation in a human subject,
which method comprises administering to said subject a composition as claimed in any one of claims 1 to 17, 19 to 32, or 35 to 59.

65. A method as claimed in claim 64 wherein the subject is a human who has been assessed as being susceptible to the disorder, optionally based on familial or genetic or other data.

66. A method as claimed in any one of claims 60 to 65 wherein the disorder is a tauopathy.

67. A method as claimed in any one of claims 60 to 66 wherein the disorder is selected from the list consisting of: Pick's disease, progressive supranuclear palsy, frontotemporal dementia, FTD with parkinsonism linked to chromosome 17, frontotemporal lobar degeneration syndromes; disinhibition-dementia-parkinsonism-amyotrophy complex, pallido-ponto-nigral degeneration, Guam-ALS syndrome, pallido nigro luysian degeneration, cortico-basal degeneration, dementia with argyrophilic grains, dementia pugilistica or chronic traumatic encephalopathy, Down's syndrome, subacute sclerosing panencephalitis, mild cognitive impairment, Niemann-Pick disease, type C, Sanfilippo syndrome type B, or a myotonic dystrophy DM1 or DM2.

68. A method as claimed in any one of claims 60 to 66 wherein the disorder is Alzheimer's disease.

69. A method as claimed in any one of claims 60 to 66 wherein the disorder is mild cognitive impairment.

70. A method as claimed in any one of claims 60 to 65 wherein the disorder is a polyglutamine disorder, such as Huntington's disease, spinal bulbar muscular atrophy, dentatorubropallidoluysian atrophy or spinocerebellar ataxias; wherein the disorder is a TDP-43 proteinopathy, such as FTLD-TDP; wherein the disorder is a synucleinopathy, such as Parkinson's disease, dementia with Lewy bodies or multiple system atrophy; wherein the disorder is hereditary cerebral angiopathy; wherein the disorder is amyotrophic lateral sclerosis; or wherein the disorder is familial encephalopathy with neuronal inclusion bodies.

71. A method as claimed in any one of claims 60 to 70 wherein the treatment is combined with a further therapeutic agent for that disorder.

72. A method as claimed in any one of claims 60 to 71, wherein the pharmaceutical composition is the solid tablet, and wherein the total daily dose is between 1 and 30 mg of MT, optionally 4-16 mg, to the subject per day, optionally split into 2 or more doses.

73. A method as claimed in claim 72 wherein the total daily dose is about 2, 3, 4, 5, 6, 7, 8, 9, 10, 11, 12, 13, 14, 15, 16, 17, 18, 19, 20, 21, 22, 23, 24, 25, 26, 27, 28, 29, or 30 mg.

74. A method as claimed in any one of claims 60 to 71, wherein the pharmaceutical composition comprises the coated capsule, and wherein the composition is not administered on consecutive days or is administered once a day.

75. A method as claimed in claim 74, wherein administration provides an amount of MT to the subject that corresponds to an average amount per day of from around any of 0.05, 0.1, 0.2, 0.3, 0.4, 0.5, 1, 1.5, and 2 mg to around any of 2.5, 3, 5, 6, 7, 8, 9, 10, 11, 12, 13, 14, 15, 16, 17, 18, 19, 20, 25 and 30 mg.

76. A method as claimed in claim 74 or claim 75, wherein administration provides an amount of MT to the subject that corresponds to an average amount per day of from around any of 0.05, 0.1, 0.2, 0.3, 0.4, 0.5, 1, 1.5, and 2 mg to around any of 2.5, 3, 5, 6, 7, 8, 9, 10, 11, 12, 13, 14, 15, 16, 17, 18, 19, 20, 25 and 30 mg.

77. A container comprising:
(i) a plurality of dosage units each of which is a composition as claimed in any one of claims 1 to 17, 19 to 32, or 35 to 59; and optionally
(ii) a label and/or instructions for their use according to a method as defined in any one of claims 60 to 76.

78. A container as claimed in claim 77, wherein the container comprises dosage units, and the dosage units are present in a blister pack which is substantially moisture-impervious.

79. A container as claimed in claim 77 or claim 78 wherein the label or instructions provide information regarding the disorder for which the composition is intended.

80. A container as claimed in any one of claims 77 to 79 wherein the label or instructions provide information regarding the maximum permitted dosage and/or the permitted frequency of dosage of the dosage units and/or the suggested duration of the treatment.

81. A DAPTZ-containing composition as defined in any one of claims 1 to 17, 19 to 32, or 35 to 59 for use in a method of treatment as defined in any one of claims 60 to 76.

82. Use of a DAPTZ-containing composition as defined in any one of claims 1 to 17, 19 to 32, or 35 to 59 in the manufacture of a medicament for use a method of treatment as defined in any one of claims 60 to 76.
